(19) United States
(12) Patent Application Publication
(10) Pub. No.: US 2019/0085292 A1
(43) Pub. Date: Mar. 21, 2019

(54) METHODS AND SYSTEMS FOR IMPROVING CELLS FOR USE IN THERAPY

(71) Applicant: The Regents of the University of California, Oakland, CA (US)

(72) Inventors: Kyriacos A. Athanasiou, Irvine, CA (US); Jerry C. Hu, Irvine, CA (US); Wendy E. Brown, Irvine, CA (US)

(21) Appl. No.: 16/137,120

(22) Filed: Sep. 20, 2018

Related U.S. Application Data
(60) Provisional application No. 62/561,076, filed on Sep. 20, 2017.

(51) Int. Cl. C12N 5/077; A61L 27/54; A61L 27/38; A61L 27/36; A61K 35/32

(52) U.S. Cl. CPC C12N 5/0655; A61L 27/54; A61L 27/3852; A61L 27/3817; C12N 2527/00; A61L 27/3691; A61K 35/32; C12N 2521/00; A61L 27/3612

(57) ABSTRACT

Methods and systems for enhancing cell populations such as chondrocytes for tissue engineering applications, e.g., for production of neocartilage. The methods and systems of the present invention feature the introduction of a hypotonic buffer to the cells during the cell isolation process, which results in neotissue (e.g., neocartilage) constructs that are significantly more mechanically robust as compared to those not treated with hypotonic buffer. The methods and systems may further comprise introducing cytochalasin D to cells purified with hypotonic buffer, which can further bolster the mechanical properties and matrix deposition of the cells. The methods and systems result in neocartilage engineered from chondrocytes, for example, from fetal aged tissue, having compressive properties on par with native adult articular cartilage.

### CROSS REFERENCE

This application claims priority to U.S. Provisional Patent Application No. 62/561,076 filed Sep. 20, 2017, the specification(s) of which is/are incorporated herein in their entirety by reference.

### GOVERNMENT SUPPORT

This invention was made with government support under Grant No. RO1 AR067821 awarded by NIH. The government has certain rights in the invention.

### FIELD OF THE INVENTION

The present invention relates to cell purification methods for use in applications such as cell and tissue engineering as well as cell and tissue transfer.

### BACKGROUND OF THE INVENTION

The goal of tissue engineering is to replace injured tissue in an effort to halt and reverse disease progression. Primary, fully differentiated cells are widely considered to be the ideal cell type for tissue engineering. They are phenotypically stable and readily produce tissue-specific extracellular matrix (ECM) molecules. Juvenile, and furthermore fetal, sources of tissue are most desirable due to their enhanced proliferative and synthetic abilities compared to adult cells. Tissue engineered products composed of juvenile cells are currently used clinically. For example, RevaFlex (ISTO Technologies), a tissue engineered product for the repair of cartilage using juvenile chondrocytes, is currently in Phase III clinical trials in the United States. While these engineered tissues show promise, they have yet to recapitulate native tissue properties and structure.

Tissue engineering efforts using primary cells may be hindered via contamination by undesirable cell types. Contamination by blood and surrounding tissue can occur during the isolation of target donor tissue. Furthermore, many tissues are composed of multiple cell types, not all of which are suitable for tissue engineering applications. Disease state and tissue maturity may additionally introduce unwanted cell phenotypes into isolated populations. Aged tissues, which are more prone to diseases such as cancer, atherosclerosis, and osteoarthritis, contain senescent cells that increasingly produce reactive oxygen species, inflammatory mediators, and matrix degrading enzymes. These limitations necessitate the use of cell purification methods during isolation to eliminate the presence of undesirable phenotypes and achieve homogeneous cell populations, enriched for cells with appropriate characteristics for tissue engineering.

Articular cartilage tissue engineering is well-established, and therefore may be used as an example system. However, not typically recognized, unwanted cell phenotypes in cartilage cells can be present due to a number of reasons. Contamination by hematopoietic cells or cells from other surrounding tissues can occur when taking cartilage biopsies in clinical applications, such as autologous chondrocyte implantation (ACI). Short term exposure of cartilage to blood has been shown to induce chondrocyte apoptosis in models reflective of hemophilia. Secondly, in a clinical setting, autologous or allogeneic cartilage grafts are often taken from adult tissues, which exhibit matrix degradation, surface defects, and fibrillation. Diseased cartilage, such as in osteoarthritis, experiences enhanced ECM degeneration and contains chondrocytes of altered phenotypes. Degenerative changes to the cartilage ECM are associated with chondrocyte apoptosis. Fetal cartilage, on the other hand, is vascularized, thus introducing blood and a plethora of cell types into the mass of tissue from which chondrocytes are isolated. Additionally, even in healthy tissue, cartilage isolation itself causes tissue damage, resulting in necrosis at the wound edge and a wave of apoptosis extending into the tissue. In addition to red blood cell (RBC) contamination, cell phenotype heterogeneity by chondrocytes of altered phenotypes is an unexpected factor limiting the ability of engineered cartilage properties from reaching those of native tissue.

Despite the potential for contamination during chondrocyte isolation, only a few studies have aimed to demonstrate its importance. Employing collagenase to sequentially digest whole hamster rib cartilage into two fractions, it was demonstrated that the second fraction contained a cell population with more homogeneous, chondrocytic morphology compared to the whole, unseparated population. Another method to purify isolated chondrocytes is via sequential plating. Rat cartilage cell isolates separated by differential adhesion to tissue culture plastic showed 100% chondrocytes after the 8^{th} plating, versus a mixture of cells when the whole population was plated. Yet another method suggests the use of cell surface markers, such as CD14 and CD45, to exclude contamination by monocytes and hematopoietic cells. Ammonium-chloride-potassium lysing buffer (ACK buffer) is commonly used to lyse RBCs in samples containing white blood cells, such as EDTA-treated whole blood, buffy coats, and bone marrow. For tissue engineering purposes, ACK buffer is used to isolate pure populations of stem cells, such as adipose-derived and mesenchymal stem cells, but has not yet been explored in the isolation of non-stem cell types. As contaminating cell types in many isolates of fully differentiated cells may include cells with alternate phenotypes, ACK buffer treatment holds promise for purification of the cell populations desirable for tissue engineering applications. Despite the potential that ACK buffer treatment lyses all cells, the present invention allows it use to preferentially destroy cells with altered phenotypes and enrich for cells with favorable phenotypes for neotissue formation.

Given the importance of cell purity, one utility of this invention is the ACK buffer treatment of freshly isolated, fully differentiated cells to enhance their capacity to form biofunctional tissues.

Without wishing to limit the present invention to any theory or mechanism, it is believed that the methods and systems of the present invention can improve the mechanical properties of neotissue made from particular cell populations (e.g., fetal-aged cells, diseased tissue sources) to those made of adult-level cells or healthy cells.

Juvenile and fetal, primary, fully differentiated cells are widely considered to be ideal cell types for tissue engineering applications. However, their use in tissue engineering may be hindered through contamination of undesirable cell types that prevent these cells of achieving functional properties similar to those made of adult-level cells or healthy cells. Increases in neocartilage mechanical properties to adult levels from fetal-aged chondrocytes have never been previously achieved.

### SUMMARY OF THE INVENTION

The present invention features methods and systems for improving cells for therapy. For example, cell purification methods that enhance cell populations by enriching for a population of cells that have characteristics conducive for cell and tissue engineering.

### Surprising Results

Because the prior art teaches that hypotonic buffer treatment is used for cell populations containing blood cells, it is surprising that cells isolated from non-vascular tissue, i.e., cartilage, respond to ACK buffer treatment.

Furthermore, the cartilage cells, which do not contain blood cells, respond to ACK buffer treatment in an unexpected way by forming engineered neocartilage, whereas the prior art instructs the use of ACK buffer treatment in stem cells.

It was surprising that subjecting cartilage cells to a hypotonic buffer, such as ACK buffer, that selects for cells that have pre-existing undesirable cytoskeletal characteristics, undesirable membrane characteristics, and altered stiffness, resulted in a population of enhanced cells.

It was surprising that there were cells with undesirable, e.g., pro-apoptotic, characteristics in young, healthy cartilage to the extent that the formation of engineered neocartilage was affected by the presence of these cells.

It was surprisingly discovered that the methods and systems of the present invention resulted in scaffold-free neocartilage engineered from the enhanced fully differentiated cells obtained from the treatments described herein achieving compressive properties on par with native adult articular cartilage. Increases in neocartilage mechanical properties to adult levels from fetal-aged chondrocytes have never before been achieved. The present invention features methods to enrich for cell populations suitable for neocartilage development and further allows for methods to manipulate the cytoskeleton to improve cells for therapy. For example, the use of a hypotonic buffer during purification of the chondrocytes resulted in significant improvements in homogeneity, matrix deposition, and mechanical properties of the neocartilage constructs. The combination of a hypotonic buffer and cytochalasin D resulted in neocartilage engineered from fetal-aged chondrocytes achieving compressive properties on par with native adult articular cartilage. Without wishing to limit this invention to any particular theory or mechanism, it is believed that in addition to reducing RBC contamination, removing chondrocytes of altered phenotypes, cellular detractors to the self-assembling process, and eliminating apoptotic stimuli improves neocartilage homogeneity, chondrocyte distribution, and ECM deposition within the neotissues, thus enhancing the biochemical and mechanical properties of engineered tissues formed with the treated cells.

These results are surprising because mechanical robustness of this level has never before been seen with fetal chondrocyte sources.

The present invention features methods of preparing cells or preparing cell populations and methods of enhancing cells populations for therapy. The present invention also features methods of preparing tissues and methods of enhancing tissues for therapy.

The present invention features a method of enhancing a cell population comprising: 1) obtaining a population of somatic cells; 2) subjecting the population of somatic cells to a treatment that selects for cells with pre-existing undesirable cytoskeletal characteristics; 3) isolating and removing cells that have pre-existing undesirable cytoskeletal characteristics; and 4) isolating and retaining the remaining cell population, enriched for cells without pre-existing undesirable cytoskeletal characteristics. These steps can be repeated multiple times, alone or in combination with other treatments.

The present invention also features a method of enhancing a cell population: 1) obtaining a population of somatic cells; 2) subjecting the population of somatic cells to a treatment that selects for cells with pre-existing undesirable membrane surface area characteristics; 3) isolating and removing cells that have pre-existing undesirable membrane characteristics; and 4) isolating and retaining the remaining cell population, enriched for cells without pre-existing undesirable membrane characteristics. These steps can be repeated multiple times, alone or in combination with other treatments.

The present invention further features a method of enhancing a cell population comprising: 1) providing a population of somatic cells; 2) subjecting the population of somatic cells to a treatment that selects for cells that have pre-existing altered stiffness characteristics; 3) isolating and removing cells that have pre-existing altered stiffness characteristics; and 4) isolating and retaining the remaining cell population, enriched for cells without pre-existing altered stiffness characteristics. These steps can be repeated multiple times, alone or in combination with other treatments.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

This patent application contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIG. 1 shows pellet morphology, viability, and red blood cell (RBC) content of fetal ovine ACs and juvenile bovine ACs before and after ACK treatment. ACK treatment resulted in a change in cell pellet color and a significant reduction in RBC content.

FIGS. 2A-2H show neocartilage gross morphology and select parameters. FIG. 2A shows that ACK treatment eliminated bulbous, diffuse regions (indicated by white arrows) in fetal ovine AC neocartilages. FIG. 2B shows that ACK treatment reduced fetal ovine neocartilage thickness. FIG. 2C shows that ACK treatment reduced fetal ovine neocartilage wet weights. FIG. 2D shows ACK treatment did not affect fetal ovine hydration. FIG. 2E shows that ACK treatment eliminated bulbous, diffuse regions (indicated by white arrows) in juvenile bovine AC neocartilages. FIG. 2F shows that ACK treatment reduced juvenile bovine neocartilage thicknesses. FIG. 2G shows that ACK treatment reduced juvenile bovine neocartilage wet weights. FIG. 2H shows that ACK treatment did not affect juvenile bovine hydration.

FIG. 3 shows neocartilage histology. ACK treatment of fetal ovine and juvenile bovine ACs eliminated the diffuse regions of low cellularity present in untreated constructs (*), enhanced neocartilage homogeneity, and intensified GAG, total collagen, and collagen II staining.

FIGS. 4A-4J show neocartilage biochemical content in fetal ovine ACs (foACs) and juvenile bovine ACs (jbACs) with and without ACK treatment. FIG. 4A shows ACK treatment significantly reduced caspase activity in foACs. FIG. 4B shows ACK treatment did not affect GAG/WW content in foACs. FIG. 4C shows ACK treatment did not affect GAG/DW content in foACs. FIG. 4D shows ACK treatment significantly increased collagen/WW content in foACs. FIG. 4E shows ACK treatment significantly increased collagen/DW content in foACs. FIG. 4F shows ACK treatment significantly reduced caspase activity in jbACs. FIG. 4G shows ACK treatment significantly reduced GAG/WW content in jbACs. FIG. 4H shows ACK treatment significantly reduced GAG/DW content in jbACs. FIG. 41 shows ACK treatment significantly increased collagen/WW content in jbACs. FIG. 4J shows ACK treatment did not affect GAG/WW content in jbACs.

FIG. 5 shows mechanical properties of neocartilage. ACK treatment significantly increased all mechanical properties measured for both cell types.

FIGS. 6A-6H show the effect of seeding density on neocartilage gross morphology, biochemical content, and histology. FIG. 6A shows that gross abnormalities appear at seeding densities of 5 and 4 million cells in P0 and P3R passages, respectively. FIGS. 6B and 6D show that GAG/DNA (FIG. 6B) and collagen/DNA (FIG. 6D) of P3R neocartilage show a seeding density-dependent effect and exceed that of P0 neocartilage. FIG. 6F shows pyridinoline content of P0 neocartilage exceeds that of P3R neocartilage. FIGS. 6C, 6E, 6G show that the mechanical properties, aggregate modulus (FIG. 6C), tensil modulus (FIG. 6E), and ultimate tensil strength (FIG. 6G), increase with seeding density of P0 cells and decrease with seeding density of P3R cells. FIG. 6H shows H & E staining and immunohistochemical (INC) staining for GAG, collagen type I (col I), collagen type II (col II), and total collagen (total col). IHC controls are meniscus (M), articular cartilage (AC), and tendon (T). (Phase 1)

FIG. 7 shows phenotypic verification of engineered neocartilage. Histology controls are articular cartilage (AC) and growth plate (GP).

FIGS. 8A-8H show the effect of cytochalasin D (Cyto D) and hyaluronidase (Hya) treatment of P3R neocartilage. FIG. 8A shows that a gross abnormality was present only in the Hya-treated group. FIGS. 8C, 8D, 8F, and 8H show GAG/wet weight (FIG. 8C) and mechanical properties, aggregate modulus (FIG. 8D), tensil modulus (FIG. 8F), and ultimate tensil strength (FIG. 8H) were increased with Cyto D treatment. FIGS. 8E and 8G show collagen (FIG. 8E) and pyridinoline (FIG. 8G) contents were unchanged with any treatment. FIG. 8B shows H&E staining and IHC staining for GAG, collagen type I (col I), collagen type II (col II), and total collagen (total col). IHC controls are meniscus (M), articular cartilage (AC), and tendon (T). (Phase 2)

FIG. 9 shows the effect of cytochalasin D treatment on actin arrangement. Cytochalasin D treatment resulted in enhanced cortical arrangement of actin within both P3 and P3R chondrocytes. (Phase 2)

FIGS. 10A-10H show the effect of Cytochalasin D (Cyto D) and TCL treatment of P3R neocartilage. FIG. 10A shows no gross abnormalities. FIG. 10B shows H&E staining and IHC staining for GAG, collagen type I (col I), collagen type II (col II), and total collagen (total col). IHC controls are meniscus (M), articular cartilage (AC), and tendon (T). FIGS. 10E and 10G show that TCL treatment in combination with Cyto D (Cyto D+TCL) increased collagen (FIG. 10E) and pyridinoline (FIG. 10G) contents. FIGS. 10F and 10H show that TCL treatment in combination with Cyto D (Cyto D+TCL) increased tensile stiffness (FIG. 10F) and strength (FIG. 10H). (Phase 3).

FIGS. 11A-11E show increases in neocartilage functional properties. FIG. 11A shows that aggregate modulus increased 9.6-fold. FIG. 11B shows that shear modulus increased 7.2-fold. FIG. 11C shows that tensile modulus increased 3.8-fold. FIG. 11D shows that the ultimate tensile strength increased 9.0-fold. FIG. 11E shows that P3R neocartilage exceeded fetal and juvenile native tissue values and approached adult levels. (Phases 1-3).

FIGS. 12A-12B show the effect of Cytochalasin D (Cyto D) and hyaluronidase (Hya) treatment of P3 Neocartilage. FIG. 12 A shows that Cyto D treatment resulted in the only flat construct. FIG. 12 B shows H&E staining and IHC staining for GAG, collagen type I (col I), collagen type II (col II), and total collagen (total col). IHC controls (B) are meniscus (M), articular cartilage (AC), and tendon (T). (Phase 2).

FIG. 13 shows Table 1 (data from Phase 1). Data are shown as mean±standard deviation. Statistics were calculated across groups within a biochemical or mechanical parameter. Statistical significance is indicated in groups marked with different letters.

FIG. 14 shows Table 2 (data from Phase 2, P3). Data are shown as mean±standard deviation. Statistics were calculated across groups within a biochemical or mechanical parameter. Statistical significance is indicated in groups marked with different letters.

FIG. 15 shows Table 3 (Phase 2). Data are shown as mean±standard deviation. Statistics were calculated across groups within a biochemical or mechanical parameter. Statistical significance is indicated in groups marked with different letters.

FIG. 16 shows Table 4 (Phase 3). Data are shown as mean±standard deviation. Statistics were calculated across groups within a biochemical or mechanical parameter. Statistical significance is indicated in groups marked with different letters.

FIG. 17 shows a summary of compressive properties. Aggregate modulus of ACK buffer treated P3R cells seeded at optimal density with cytochalasin D was increased 9.6-fold over the P0 control.

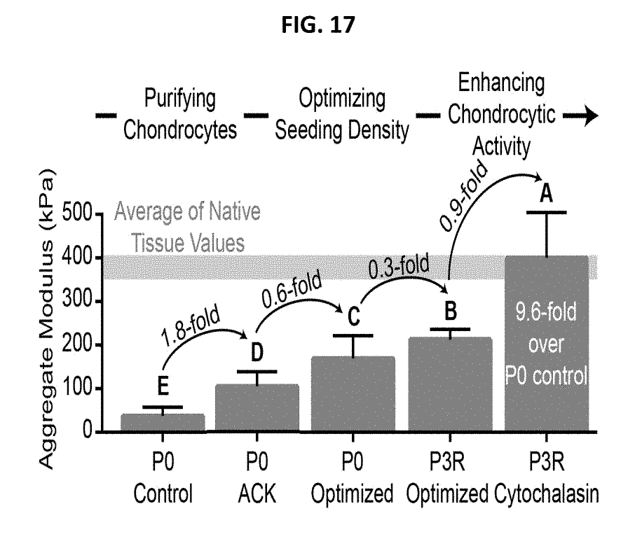

### DETAILED DESCRIPTION OF THE INVENTION

The present invention features methods and systems for improving cells for therapy, for example, cell purification methods that enhance cell populations. The cells are used for tissue engineering applications and for cell or tissue transfer. Cell populations may comprise fully differentiated cells, such as chondrocytes, osteoblasts, adipocytes, cardiomyocytes. Tissues may comprise fat, cartilage, bone, tendons, ligaments, muscle, skin.

Enhancement of the cell population is considered to be improved homogeneity of cells with characteristics suitable for cell/tissue engineering, improved robustness of cells, improved cell phenotype, improved characteristics that lead to improvements in tissue engineering for example, faster production of neotissue or better neotissue constructs.

The present invention features methods comprising 1) isolating cells or tissue, e.g., from a donor or a source and 2) chemically or physically/mechanically treating the cells (e.g., chondrocytes). A non-limiting example of a chemical treatment comprises the introduction of a hypotonic buffer to the cells during the cell purification process resulting in neotissue constructs (e.g., neocartilage) that are significantly more mechanically robust. The method may comprise pelleting the cells.

The present invention features purification methods based on characteristics of cells comprising cytoskeletal, membrane surface area, and stiffness properties. Without wishing to limit this invention to any particular theory or mechanism, it is believed that the purification treatment preferentially selects for cells with pre-existing undesirable characteristics or cells with altered phenotype (compromised cells), including but not limited to fragmented cytoskeleton, reduced membrane surface area, and altered cell stiffness. These compromised cells are removed, resulting in an enriched cell population for cells with characteristics conducive for functional cell and neotissue development.

In some embodiments, the cells that are removed by the treatment comprise one or more percent of the population of cells or tissues from cartilage, wherein the removed cells are designated to have pre-existing undesirable cytoskeletal, membrane surface area, and/or stiffness properties. The population of cells or tissues being used in accordance with the present invention may be cells freshly extracted from a cartilage from a living subject, or cells that have been previously frozen or otherwise preserved, or cells that have been previously in culture in vitro or in vivo,

In some embodiments, the cells with pre-existing undesirable cytoskeletal characteristics comprise cells with weakened, fragmented, disrupted or modified cytoskeletons, cells with cytoskeletons that are unable to remodel or have reduced remodeling ability, cells with a cytoskeletal properties that render cells more susceptible to destruction by the treatment, or a combination thereof.

Without wishing to limit the invention to any particular theory or mechanism, it is believed that at least 1% of a cell population (e.g., chondrogenic cell population) has pre-existing undesirable cytoskeletal characteristics. Thus, in some embodiments, the treatment using chemical or physical methods (e.g., swelling, shearing, compression) targets to eliminate at least 1% (but less than 99%) of a cell population (e.g., chondrogenic cell population) to ensure the elimination of cells (e.g., chondrocytes) with pre-existing undesirable cytoskeletal properties. For example, screening conditions may be set as to cause an elimination of at least 1% (but less than 99%) of a cell population based on their pre-existing undesirable cytoskeletal characteristics. In some embodiments, the treatment targets to eliminate at least 5% of a cell population to ensure the elimination of cells with pre-existing undesirable cytoskeletal properties. In some embodiments, the treatment targets to eliminate at least 10% of a cell population to ensure the elimination of cells with pre-existing undesirable cytoskeletal properties. In some embodiments, the treatment targets to eliminate at least 15% of a cell population to ensure the elimination of cells with pre-existing undesirable cytoskeletal properties. In some embodiments, the treatment targets to eliminate at least 20% of a cell population to ensure the elimination of cells with pre-existing undesirable cytoskeletal properties. In some embodiments, the treatment targets to eliminate at least 25% of a cell population to ensure the elimination of cells with pre-existing undesirable cytoskeletal properties. In some embodiments, the treatment targets to eliminate at least 30% of a cell population to ensure the elimination of cells with pre-existing undesirable cytoskeletal properties. In some embodiments, the treatment targets to eliminate at least 35% of a cell population to ensure the elimination of cells with pre-existing undesirable cytoskeletal properties. In some embodiments, the treatment targets to eliminate at least 40% of a cell population to ensure the elimination of cells with pre-existing undesirable cytoskeletal properties. In some embodiments, the treatment targets to eliminate at least 45% of a cell population to ensure the elimination of cells with pre-existing undesirable cytoskeletal properties. In some embodiments, the treatment targets to eliminate at least 50% of a cell population to ensure the elimination of cells with pre-existing undesirable cytoskeletal properties. In some embodiments, the treatment targets to eliminate at least 55% of a cell population to ensure the elimination of cells with pre-existing undesirable cytoskeletal properties. In some embodiments, the treatment targets to eliminate at least 60% of a cell population to ensure the elimination of cells with pre-existing undesirable cytoskeletal properties. In some embodiments, the treatment targets to eliminate at least 65% of a cell population to ensure the elimination of cells with pre-existing undesirable cytoskeletal properties. In some embodiments, the treatment targets to eliminate at least 70% of a cell population to ensure the elimination of cells with pre-existing undesirable cytoskeletal properties. In some embodiments, the treatment targets to eliminate at least 75% of a cell population to ensure the elimination of cells with pre-existing undesirable cytoskeletal properties.

In some embodiments, the cells with undesirable membrane characteristics comprise cells with reduced membrane surface area, cells with a disrupted or modified membrane, cells with membrane unable to adjust to conformational changes/change in size, cells with membrane properties that render the cells more susceptible to destruction by the treatment, or a combination thereof.

Without wishing to limit the invention to any particular theory or mechanism, it is believed that at least 1% of a cell population (e.g., chondrogenic cell population) has pre-existing undesirable membrane surface area properties. Thus, in some embodiments, the treatment using chemical or physical methods (e.g., swelling, shearing, compression) targets to eliminate at least 1% (but less than 99%) of a cell population (e.g., chondrogenic cell population) to ensure the elimination of cells (e.g., chondrocytes) with pre-existing undesirable membrane surface area properties. For example, screening conditions may be set as to cause an elimination of at least 1% (but less than 99%) of a cell population based on their pre-existing undesirable membrane surface area characteristics. In some embodiments, the treatment targets to eliminate at least 5% of a c cell population to ensure the elimination of cells with pre-existing undesirable membrane surface area properties. In some embodiments, the treatment targets to eliminate at least 10% of a cell population to ensure the elimination of cells with pre-existing undesirable membrane surface area properties. In some embodiments, the treatment targets to eliminate at least 15% of a cell population to ensure the elimination of cells with pre-existing undesirable membrane surface area properties. In some embodiments, the treatment targets to eliminate at least 20% of a cell population to ensure the elimination of cells with pre-existing undesirable membrane surface area properties. In some embodiments, the treatment targets to eliminate at least 25% of a cell population to ensure the elimination of cells with pre-existing undesirable membrane surface area properties. In some embodiments, the treatment targets to eliminate at least 30% of a cell population to ensure the elimination of cells with pre-existing undesirable membrane surface area properties. In some embodiments, the treatment targets to eliminate at least 35% of a cell population to ensure the elimination of cells with pre-existing undesirable membrane surface area properties. In some embodiments, the treatment targets to eliminate at least 40% of a cell population to ensure the elimination of cells with pre-existing undesirable membrane surface area properties. In some embodiments, the treatment targets to eliminate at least 45% of a cell population to ensure the elimination of cells with pre-existing undesirable membrane surface area properties. In some embodiments, the treatment targets to eliminate at least 50% of a cell population to ensure the elimination of cells with pre-existing undesirable membrane surface area properties. In some embodiments, the treatment targets to eliminate at least 55% of a cell population to ensure the elimination of cells with pre-existing undesirable membrane surface area properties. In some embodiments, the treatment targets to eliminate at least 60% of a cell population to ensure the elimination of cells with pre-existing undesirable membrane surface area properties. In some embodiments, the treatment targets to eliminate at least 65% of a cell population to ensure the elimination of cells with pre-existing undesirable membrane surface area properties. In some embodiments, the treatment targets to eliminate at least 70% of a cell population to ensure the elimination of cells with pre-existing undesirable membrane surface area properties. In some embodiments, the treatment targets to eliminate at least 75% of a cell population to ensure the elimination of cells with pre-existing undesirable membrane surface area properties.

In some embodiments, the cells with undesirable stiffness characteristics comprise cells with reduced overall stiffness, cells with increased overall stiffness, cells with stiffness which varies depending on the region of the cell tested, cells with reduced pliability, cells with stiffness properties that render the cells more susceptible to destruction by the treatment, or a combination thereof.

Without wishing to limit the invention to any particular theory or mechanism, it is believed that at least 1% of a cell population (e.g., chondrogenic cell population) has pre-existing undesirable stiffness properties. Thus, in some embodiments, the treatment using chemical or physical methods (e.g., swelling, shearing, compression) targets to eliminate at least 1% (but less than 99%) of a cell population (e.g., chondrogenic cell population) to ensure the elimination of cells (e.g., chondrocytes) with pre-existing undesirable stiffness properties. For example, screening conditions may be set as to cause an elimination of at least 1% (but less than 99%) of a cell population based on their pre-existing undesirable stiffness characteristics. In some embodiments, the treatment targets to eliminate at least 5% of a cell population to ensure the elimination of cells with pre-existing undesirable stiffness properties. In some embodiments, the treatment targets to eliminate at least 10% of a cell population to ensure the elimination of cells with pre-existing undesirable stiffness properties. In some embodiments, the treatment targets to eliminate at least 15% of a cell population to ensure the elimination of cells with pre-existing undesirable stiffness properties. In some embodiments, the treatment targets to eliminate at least 20% of a cell population to ensure the elimination of cells with pre-existing undesirable stiffness properties. In some embodiments, the treatment targets to eliminate at least 25% of a cell population to ensure the elimination of cells with pre-existing undesirable stiffness properties. In some embodiments, the treatment targets to eliminate at least 30% of a cell population to ensure the elimination of cells with pre-existing undesirable stiffness properties. In some embodiments, the treatment targets to eliminate at least 35% of a cell population to ensure the elimination of cells with pre-existing undesirable stiffness properties. In some embodiments, the treatment targets to eliminate at least 40% of a cell population to ensure the elimination of cells with pre-existing undesirable stiffness. In some embodiments, the treatment targets to eliminate at least 45% of a cell population to ensure the elimination of cells with pre-existing undesirable stiffness properties. In some embodiments, the treatment targets to eliminate at least 50% of a cell population to ensure the elimination of cells with pre-existing undesirable stiffness properties. In some embodiments, the treatment targets to eliminate at least 55% of a cell population to ensure the elimination of cells with pre-existing undesirable stiffness properties. In some embodiments, the treatment targets to eliminate at least 60% of a cell population to ensure the elimination of cells with pre-existing undesirable stiffness properties. In some embodiments, the treatment targets to eliminate at least 65% of a cell population to ensure the elimination of cells with pre-existing undesirable membrane surface area properties. In some embodiments, the treatment targets to eliminate at least 70% of a cell population to ensure the elimination of cells with pre-existing undesirable stiffness properties. In some embodiments, the treatment targets to eliminate at least 75% of a cell population to ensure the elimination of cells with pre-existing undesirable stiffness properties.

In appropriate circumstances, purification comprises subjecting the population of cells to a treatment that 1) induces cell swelling; 2) induces shearing; 3) applies impact or compression; or combination thereof.

Non-limiting examples of methods that induce cell swelling comprise adding a hypotonic buffer (e.g., ACK buffer), performing freeze-thaw cycles, applying decompression of dissolved gasses, applying a vacuum or negative pressure, or applying a combination thereof.

Examples of methods that induce shearing include but not limited to fluid flow shearing, opposing microfluidic flow, forcing cells through a small filter/mesh or pathway/tunnel at high pressure, nebulizing the solution, or combination thereof.

Non-limiting examples of methods that impact or induce compression comprise forcing through a small filter/mesh or pathway/tunnel at high pressure, applying mechanical compression, applying physical collisions, or combination thereof.

In some embodiments, purification methods further comprise treating the cells with high frequency oscillations, for example treating with sonication or creating cavitation.

In some embodiments, the hypotonic buffer comprises ammonium chloride potassium (ACK) buffer. The ACK buffer may have a formula such as 154 mM ammonium chloride, 10 mM potassium bicarbonate, 97 μM EDTA, however the ACK buffer is not limited to this formula. In appropriate circumstances, the hypotonic buffer comprises Gey's buffer, Tris-HCI, HEPES +EGTA +MgCI, MP-40 lysis buffer, RIPA lysis buffer, SDS, hypotonic saline, diluted PBS, purified water, or a combination thereof. The present invention is not limited to the aforementioned hypotonic buffers.

Isolating the cells from the donor or source may comprise obtaining tissue from the donor, digesting the tissue with enzymes comprising collagenase, dispase, pronase, or a combination thereof, filtering cells from the tissue digested with enzymes, and resuspending the cells in a buffer (e.g., the hypotonic buffer or an alternative buffer) or culture medium.

Any appropriate cell population may be used. For example, the cells may be mammalian cells or plant cells. In some embodiments, the cells comprise chondrocytes (e.g., primary chondrocytes), osteoblasts, cardiomyocytes, adipocytes, hepatocytes, tenocytes, osteoclasts, smooth muscle cells, pericytes, neural cells, fibroblasts, keratinocytes, endothelial cells, myocytes, mesenchymal stem cells, hematopoietic stem cells, adipose-derived stem cells, or a combination thereof. In some embodiments, the population of cells are a combination of cell types. The present invention is not limited to the aforementioned cell types or cell origins.

In some embodiments, the cells are healthy cells. In some embodiments, the cells are from diseased tissues or sources (e.g., osteoarthritic cartilage).

The methods of the present invention further comprise introducing a cytoskeleton-modifying agent, an actin polymerization inhibitor (e.g., cytochalasin D), and/or cytoskeleton polymerization modifiers (e.g., inhibitors or enhancers, e.g., an inhibitor of polymerization of microtubules) to cells already purified with the aforementioned hypotonic buffer. The cytoskeleton modifying agent and/or actin polymerization inhibitor and/or cytoskeleton polymerization modifier may further bolster the mechanical properties and matrix deposition of the cells. The present invention is not limited to cytochalasin D.

In some embodiments, the cytoskeleton modifying agent and/or actin polymerization inhibitor and/or cytoskeleton polymerization modifier comprises microfilament or actin stabilizers, polymerizers, or polymerization inhibitors (e.g., cytochalasin family, alternative cytochalasin, latrunculin, jasplakinolide, phalloidin, swinholide, colchicine), intermediate filament stabilizers, polymerizers, or polymerization inhibitors, microtube stabilizers, polymerizers, or polymerization inhibitors, lysophosphatidic acid, staurosporine, blebbistatin, Y27632, septins, and combinations thereof. These agents (cytoskeleton modifying agent and/or actin polymerization inhibitor and/or cytoskeleton polymerization modifier) are compounds that act directly or indirectly on the cytoskeleton (e.g., Y27632, which acts upstream in a signaling cascade to affect myosin function). As a non-limiting example, the addition of cytochalasin D may improve the mechanical properties and matrix deposition of neocartilage engineered with hypotonic buffer-purified, multiple-passaged chondrocytes. The present invention is not limited to the aforementioned compounds.

The method may further comprise treating the cells with a cytoskeleton modifying agent, an actin polymerization inhibitor (e.g., cytochalasin D), a cytoskeleton polymerization modifier, or a combination thereof before treating the cells with hypotonic buffer.

In some embodiments, the cytoskeleton modifying agent, the actin polymerization inhibitor, or the cytoskeleton polymerization modifier act directly or indirectly upstream in a signaling cascade. The cytoskeleton modifying agents inhibits, stabilizes, or enhances the cytoskeleton.

In some embodiments, cytochalasin D (or the cytoskeleton modifying agent, actin polymerization inhibitor, and/or cytoskeleton polymerization modifier) is applied at 0-48 hours during neocartilage formation.

In some embodiments, the hypotonic buffer is introduced after cell isolation from tissue, after thawing, after monolayer expansion, after re-differentiation, or before neotissue formation. The hypotonic buffer may be applied to the tissue using a mechanical means or perfusion.

The method of treating the subject may comprise using the isolated, retained cells directly for therapy.

The method may comprise further subjecting the isolated, retained cells to culture in two dimensions with monolayer passaging to any extent.

The method may comprise further subjecting the isolated, retained cells to culture in three dimensions comprising one or more of the following: 1) suspension culture; 2) with scaffolds of any shape or size such as hydrogels, collagen gels, alginate, de-cellularized membranes or tissues, dehydrated membranes or tissues, freeze-dried membranes or tissues, ceramics such as hydroxyapatite of all stoichiometries, α-tricalcium phosphate, β-tricalciumphosphate, natural matrices such as silk, synthetic materials such as Poly(lactic acid) or polylactic acid or polylactide (PLA), poly(lactic-co-glycolic acid) (PLGA), Polyethylene glycol (PEG), Polyglycolide (PGA), polycaprolactone, or combinations thereof; 3) scaffold-free techniques such as self-assembly, pellet culture, aggregate culture, cell sheets, tissue fusion, or combinations of any of those; 4) combinations of scaffold-free and scaffold-based; 5) alone or with cells of other types and treatments

The method may further comprise seeding the isolated, retained cells (e.g., after pelleting). The cells may be seeded in a non-adherent well. The method may further comprise seeding the cells (e.g., chondrocytes), e.g., after pelleting, in a non-adherent well, wherein the cells seeded into the non-adherent well form neocartilage. The present invention is not limited to seeding cells in a non-adherent well.

In some embodiments, the resulting neocartilage has increased mechanical properties (e.g., one or more of: aggregate modulus, shear modulus, tensile modulus, compressive stiffness, tensile stiffness, and tensile strength) as compared to neocartilage made from chondrocytes that are not treated with hypotonic buffer.

In some embodiments, the neocartilage improves neocartilage matrix synthesis and deposition as compared to neocartilage made from chondrocytes that are not treated with hypotonic buffer. In some embodiments, the neocartilage may improve collagen crosslinking as compared to neocartilage made from chondrocytes that are not treated with hypotonic buffer.

In some embodiments, the donor is a fetal donor, a juvenile donor, or an adult donor.

The method may comprise further subjecting the isolated, retained cells to chemical factors or bioactive agents. Non-limiting examples of these factors and agents comprise active and latent forms of growth factors (e.g., TGF superfamily, growth differentiation factors, bone morphogenetic proteins), cytoskeletal modifying agents (cytochalasin D), bioactive agents, hormones (e.g., triiodothyronine, parathyroid hormone), mitogens, enzymes (e.g., chondroitinase-ABC, lysyl oxidase, lysl oxidase, lysl oxidase-like 2), collagen crosslinking agents, toxic compounds, molecules that act upstream in a signaling cascade, or a combination thereof.

The method may comprise further subjecting the isolated, retained cells to molecules comprising one or more SZP/PRG4, chondroitin sulfate, link protein, hyaluronan, keratin sulfate, dermatan sulfate, and aggrecan, collagens of type I, II, III, V, VI, X, and XI, or any agents that increase the production of these molecules.

The method may comprise further subjecting the isolated, retained cells to varying oxygen tensions achieved by environmental oxygen deprivation or enzymatic conditions.

The method may further comprise treating the cells with a physical stimulus, e.g., static or dynamic direct compression, hydrostatic pressure, shear, tension, fluid flow-induced shear, perfusion, or a combination thereof.

The method may further comprise treating the isolated, retained cells with hyaluronidase in combination with the cytoskeleton modifying agent, the actin polymerization inhibitor, or the cytoskeleton polymerization modifier.

The method of the present invention enhances the cell population. The method may improve the homogeneity of the cells. The method may improve the robustness of the cell population.

The method may further comprise using the isolated, retained cells in combinations of other prepared cells and tissues.

The method may be applied to cells or tissue for the purposes of tissue engineering, such as, for example, cartilage tissue engineering. The method may be applied to the cells or tissue for the purposes of cell transfer, such as, for example, autologous chondrocyte implantation (ACI). The method may be applied to the tissue for the purposes of tissue transfers, such as, for example, mosaicplasty.

Without wishing to limit the present invention to any theory or mechanism, it is believed that the methods and systems of the present invention can improve the mechanical properties of neotissue made from fetal-aged cells to those made of adult-level cells. Without wishing to limit the present invention to any theory or mechanism, it is believed that the methods and systems of the present invention are advantageous because there are currently no standardized chondrocyte purification methods.

The present invention is not limited to cells for use in engineering applications. For example, the methods and systems of the present invention may be used for a variety of different applications, e.g., cancer cell applications, cell purification processes, grafting (e.g., fat grafting). In some embodiments, the present methods of enhancing cell populations provide a desirable population of cells that is used prior to or in preparation for treating a subject. The enhanced cells can be directly administered to the subject (post enhancement use). The enhanced cells can be further cultured in vitro in two dimensions, including passaging in monolayer (post enhancement use), prior to administering to a subject. The enhanced cells can be further cultured in vitro in three dimensions, including suspension culture (post enhancement use), prior to administering to a subject. The enhanced cells can be further cultured in vitro for tissue engineering using scaffold-free systems, including self-assembly, or using scaffold-based systems, including natural and synthetic materials (post enhancement use), prior to administering to a subject. The enhanced cells can be used for cell transfer, tissue transfer, and/or grafting for treating a subject (post enhancement use). The enhancements methods may be followed by one or more of these post enhancement uses.

The present invention is not limited to cells for use in engineering applications. For example, the methods and systems of the present invention may be used for a variety of different applications, e.g., cancer cell applications, cell purification processes, grafting (e.g., fat grafting).

The hypotonic buffer may be introduced at any point in culture, such as after monolayer expansion, after redifferentiation, or before neotissue formation to create an enriched population of cells free of cells with pre-existing undesirable cytoskeleton, membrane surface area, and stiffness characteristics. As previously discussed the present invention is not limited to ACK buffer.

As previously discussed, a cytoskeleton modifying agent and/or actin polymerization inhibitor (e.g., cytochalasin D) and/or cytoskeleton polymerization modifier may be optionally applied. Example 4 below describes cytochalasin D application. As an example, in some embodiments, 2 μM cytochalasin D may be applied at 0-48 hours during neocartilage formation via the self-assembling process. Note the present invention is not limited to Example 4; cytochalasin D may be used with other cartilage tissue engineering systems, such as but not limited to self-organization or scaffold-based systems, as well as other sources of chondrocytes, such as nasal or ear chondrocytes or osteoarthritic chondrocytes.

The methods described herein may be used independently or in combination. Application of the purification treatment (e.g., hypotonic buffer) and/or cytoskeleton modifying agent(s) may be applied at different time points throughout the culture.

The present invention also features tissue engineering of various tissues such as articular cartilage using purified cells, or cell transfer, or fat grafting. In some embodiments, the pelleted cells are for cell transfer or for tissue engineering, or for grafting. In some embodiments, the pelleted cells are for cell injection.

In some embodiments, the method of the present invention comprises isolating cells from a donor; treating the cells with hypotonic buffer; pelleting the cells; passaging/expanding the cells in monolayer re-differentiating the cells, and seeding the re-differentiated cells. The cells can be seeded in a non-adherent well (e.g., non-adherent agarose well). The present invention is not limited to seeding cells in a non-adherent well. Technologies for tissue engineering may be scaffold-based or scaffold-free.

In some embodiments, the methods of the present invention are for preparing neotissue made from fetal-aged chondrocytes having mechanical properties similar to those of adult articular cartilage.

The methods may be for enriching for populations of cells that have pre-existing characteristics conducive for functional cells and/or neotissue formation, including but not limited to cells with intact cytoskeleton able to remodel, cells with high membrane surface area, and cells with unaltered stiffness (cells able to make conformational changes). The methods may be for improving a population of cells to engineer native-like neocartilage. The methods may be for improving a population of cells to engineer native-like neotissue.

In some embodiments, the methods of the present invention allow for the use of a lower seeding density (e.g., for neotissue production), e.g., the methods of the present invention improve robustness of the cell population such that fewer cells are needed (e.g., as compared to other methods). In some embodiments, a seeding density of about 2 million cells per construct is used. In some embodiments, using a seeding density of about 2 million cells per construct further increases aggregate modulus and shear modulus.

Note that in the present invention, additional biochemical treatments and/or mechanical stimuli may be used in combination with (i) a hypotonic buffer; (ii) a cytoskeleton modifying agent, an actin polymerization inhibitor (e.g., cytochalasin D), a cytoskeleton polymerization modifier, or a combination thereof; or (iii) both the hypotonic buffer and the cytoskeleton modifying agent, actin polymerization inhibitor (e.g., cytochalasin D), cytoskeleton polymerization modifier, or a combination thereof. For example, the present invention may feature: (A) the use of a hypotonic buffer to prepare cells for cell transfer and/or tissue engineering in a scaffold-free or scaffold-based system: (i) preparation may include the use of a physical stimulus (e.g., shear), (ii) preparation may feature additional treatment with a biochemical treatment, (iii) preparation may feature additional stimuli with mechanical means, (iv) preparation may feature additional treatment and stimulation with biochemical and mechanical means; (B) the use of cytochalasin D to enhance engineered neocartilage (both scaffold-free and scaffold-based systems): (i) preparation may feature additional treatment with biochemical treatments; (ii) preparation may feature additional stimuli with mechanical means; (iii) preparation may feature additional treatment and stimulation with biochemical and mechanical means; and (C) the use of hypotonic buffer and cytochalasin D together: (i) preparation may feature additional treatment with biochemical treatments; (ii) preparation may feature additional stimuli with mechanical means; (iii) preparation may feature additional treatment and stimulation with biochemical and mechanical means.

In summary, non-limiting examples of the present invention comprise (1) hypotonic buffer; (2) cytochalasin D; (3) hypotonic buffer+cytochalasin D; (4) hypotonic buffer+biochemical treatment; (5) hypotonic buffer+physical stimulus; (6) hypotonic buffer+biochemical treatment+physical stimulus; (7) cytochalasin D+biochemical treatment; (8) cytochalasin D+physical stimulus; (9) cytochalasin D+biochemical treatment+physical stimulus; (10) hypotonic buffer+cytochalasin D+biochemical treatment; (11) hypotonic buffer+cytochalasin D+physical stimulus; (12) hypotonic buffer +cytochalasin D +biochemical treatment +physical stimulus. Note that cytochalasin D as mentioned above may be replaced with a cytoskeleton modifying agent, an actin polymerization inhibitor, a cytoskeleton polymerization modifier, or a combination thereof.

The methods and systems of the present invention (e.g., use of hypotonic buffer, use of a cytoskeleton modifying agent and/or actin polymerization inhibitor and/or cytoskeleton polymerization modifier) may be used independently or in conjunction with each other, or in conjunction with other bioactive agents (for example, growth factors, chondroitinase ABC, lysyl oxidase like 2) and physical/mechanical stimuli (for example, direct compression, shear, hydrostatic pressure, tension.) e.g., to achieve greater functional properties of engineered neotissues (e.g., articular cartilage).

Without wishing to limit the present invention to any theory or mechanism, it is believed that treatment with a cytoskeleton modifying agent and/or actin polymerization inhibitor (e.g., cytochalasin D) and/or cytoskeleton polymerization modifier is advantageous because it helps elicit native-like compressive properties in engineered neocartilage. Specifically, multiple-passaged fetal chondrocytes treated with cytochalasin D while undergoing self-assembly formed neocartilage with compressive properties on par with native adult cartilage; mechanical robustness of this level has never before been seen with fetal chondrocyte sources.

The Examples below describe the application of ACK buffer to chondrocyte isolates from fetal ovine and juvenile bovine sources. This treatment resulted in significant improvements in homogeneity, matrix deposition, and mechanical properties of the neocartilage constructs.

Without wishing to limit the present invention to any particular theory or mechanism, it is believed that purification processes are effective at increasing functionality of cells for therapy by reducing contaminating cells, particularly reducing the population of cells that have pre-existing undesirable characteristics of compromised cells, including but not limited to cells with a weakened cytoskeleton, cells with low membrane surface area, and cells with high stiffness.

The present invention is not limited to the methods or compositions described herein.

A. Purification Based on Cytoskeletal Properties

### EXAMPLE 1

### Hypotonic Solution

Example 1 describes methods of using a hypotonic solution to select cells based on cytoskeletal properties. Example 1 shows that treatment with the hypotonic solution, ACK buffer, of freshly isolated, fully differentiated cells, enhances their capacity to form biofunctional tissues. Clinically relevant articular chondrocytes (ACs) from fetal and juvenile cartilage were used as the model in the following studies: Fetal ovine articular chondrocytes (foACs) were treated with ACK buffer during their isolation. Without wishing the invention to any particular theory or mechanism, it is believed that treatment of cartilage cells with a hypotonic buffer is effective to increase viable chondrocyte purity by reducing the number of cells with pre-existing undesirable cytoskeletal characteristics. Therefore, this treatment produces a population of cells, enriched for viable chondrocytes without undesirable cytoskeletal characteristics, thereby increasing the functional properties of the resulting self-assembling neocartilage. The effects of ACK buffer treatment were also examined on cells from an animal model of different species and age, specifically juvenile bovine articular chondrocytes (jbACs).

Cell isolation: foACs were harvested from the patellofemoral surfaces of the stifle joints of three fetal (120-125-day gestation), female, Dorper cross sheep. jbACs were harvested from the patellofemoral surfaces of the stifle joints of three juvenile (2-14 days), male, Holstein and Jersey calves. Processing of ovine and bovine tissues was the same. Articular cartilage from the whole surface of both condyles and the trochlear groove were minced into approximately 1 mm³ pieces, then washed and centrifuged (500 G for 5 minutes) three times with Dulbecco's Modified Eagle Medium containing 4.5 g/L glucose and GlutaMAX (DMEM; Gibco) and 2% (v/v) penicillin/streptomycin/fungizone (PSF; BD Biosciences). The tissue was digested in 0.2% (w/v) collagenase type II (Worthington) in DMEM containing 3% (v/v) fetal bovine serum (FBS; Atlanta Biologicals) for 18 hours at 37° C. with gentle rocking. After digestion, the resultant cell solutions were filtered through 70 μm cell strainers, centrifuged (500 G for 5 minutes), and resuspended in blank DMEM. AC and RBCs were counted and the viability of ACs was assessed by Trypan Blue staining. Half of the foACs and half of the jbACs were treated with ACK buffer, as described in detail below. Cells were counted and viability was assessed again after ACK buffer treatment. Untreated cells were washed with blank DMEM instead of ACK buffer, but were otherwise handled the same way. Cells immediately underwent self-assembly.

ACK buffer treatment: The ACK buffer consisted of 154.4 mM ammonium chloride (Sigma), 10 mM potassium bicarbonate (Sigma-Aldrich), 97.3 μM ethylenediaminetetraacetic acid (EDTA) tetrasodium salt (Acros Organics). This corresponds to 8.26 g ammonium chloride, 1.0 g potassium bicarbonate, and 0.037 g EDTA in 1L of ultrapure water. This solution was sterile filtered before use.

Protocol for introducing ACK buffer to purify chondrocytes: (1) Warm ACK buffer to 37° C. (2) Portion up to 100 million chondrocytes into a 50 mL conical tube. (3) Centrifuge the cell solution at 500 G for 5 minutes. (4) Aspirate the supernatant and gently resuspend the cell pellet in 10 mL of ACK buffer. Incubate for 3-5 minutes at 37° C. (5) Centrifuge the ACK buffer cell suspension at 500 G for 5 minutes. (6) Aspirate the ACK buffer. Wash the cell pellet twice with blank or washing medium before plating or freezing.

Neocartilage construct seeding and culture: Primary foACs and jbACs treated with ACK buffer (+ACK Treatment) and untreated (−ACK Treatment) were each self-assembled into engineered neocartilage constructs in non-adherent agarose wells. A sterile stainless-steel mold consisting of 5 mm diameter cylindrical posts was inserted into a 48 well plate, each well containing 1 mL molten 2% (w/v) molecular biology grade agarose (Thermo) to create a single agarose well in each plate well. After solidification of the agarose at room temperature, the mold was removed. Agarose wells were filled with chemically defined chondrogenic medium (CHG medium) (DMEM containing 1% PSF, 1% ITS+premix (BD Biosciences), 1% non-essential amino acids (Gibco), 100 nM dexamethasone (Sigma), 50 mg/mL ascorbate-2-phosphaste (Sigma), 40 g/mL L-proline (Sigma), and 100 mg/mL sodium pyruvate (Sigma). CHG medium was exchanged twice over the course of 5 days to ensure saturation of the agarose before cell seeding. Treated and untreated foACs and jbACs were each seeded at 4.5 million cells per construct into 5 mm agarose wells in 100 μL CHG medium. Constructs were unconfined at day 6 and placed in larger wells coated with agarose to prevent construct adhesion to the wells. Medium was exchanged daily prior to unconfinement and every other day after for the duration of the 6-week culture period. Gross morphological analysis, histology, immunohistochemistry (INC), quantification of glycosaminoglycans (GAGs) and collagen, and mechanical evaluation were performed at the end of the culture period.

Gross morphological analysis: Construct thickness was measured from pictures of the constructs using ImageJ software (National Institutes of Health). Whole constructs were weighed to obtain wet weights before samples were portioned for histological, biochemical, and mechanical analysis.

Histological and immunohistochemical (INC) evaluation: Samples were fixed in 10% neutral buffered formalin, embedded in paraffin, and sectioned along the short axis into 5 μm sections to expose the full thickness of the construct. Sections were stained with Hematoxylin and Eosin (H&E) to show morphology, Safranin O/Fast Green to visualize GAGs, and Picrosirius Red to visualize collagen. Additionally, IHC was performed for collagen I (ab90395, dilution 1:250, Abcam) and collagen II (ab34712, 1:4000 dilution, Abcam).

Biochemical evaluation: Construct samples portioned for biochemical analysis were weighed to measure wet weights, lyophilized, and weighed again to measure dry weights. Construct hydration was by normalizing the difference in weights before and after lyophilization to the sample wet weight. Lyophilized samples were digested in 125 μg/mL papain (Sigma-Aldrich) at 65° C. for 18 hours. GAG content was quantified by a Blyscan assay kit (Biocolor). Collagen content was quantified by a modified colorimetric chloramine-T hydroxyproline assay. A standard curve was generated using a Sircol collagen standard (Biocolor). DNA content was quantified with PicoGreen dsDNA reagent (Invitrogen). Both collagen and GAG contents were normalized to wet weight, dry weight, and DNA content.

Mechanical evaluation: Creep indentation compressive testing was performed on 3 mm diameter punches from each construct. A 0.8 mm diameter, flat, porous indenter tip was applied to the samples using masses ranging from 0.45 to 2 g to achieve 10-15% strain. A semi-analytical, semi-numerical, linear biphasic model and a finite element model were used to obtain the aggregate and shear moduli from the experimental data. For tensile testing, samples were punched into dog bone-shaped specimens with gauge lengths of 1.92 mm, adherent to ASTM standards (ASTM D3039). Paper tabs were glued to the samples outside the gauge length, gripped in a TestResources machine (TestResources Inc.), and pulled at 1% of the gauge length per second until sample failure. The cross-sectional area of samples was measured with ImageJ and used to generate a stress-stain curve. The tensile modulus was obtained by a least-squares fit of the linear region of the curve. The maximum stress yielded the ultimate tensile strength (UTS).

Statistical analysis: A Student's t-test in Prism 6 (GraphPad Software) was used to analyze the biochemical and mechanical data. A p-value of <0.05 indicated statistical significance. A sample size of n=6 per group was used. In figures displaying quantitative results, groups not marked by the same symbol are statistically different. All data are presented as means±standard deviations.

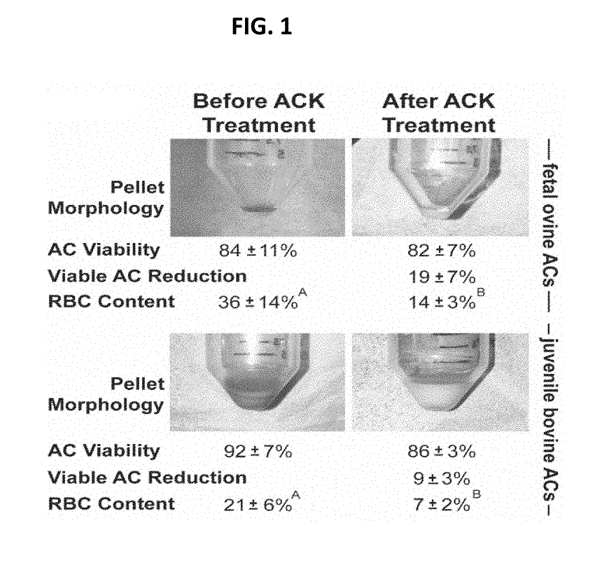

Results: FIG. 1 shows the isolated cell pellet morphology and cell counts before and immediately after ACK buffer treatment. ACK buffer treatment resulted in a morphological change of the pellets of both cell types. The foAC pellet before treatment appeared light red throughout and milky white after treatment. The jbAC pellet appeared tan with a pink cast before treatment and milky white after treatment. Viability of foACs before and after treatment was 84±11° A and 82±7%, respectively. Viability of jbACs before treatment was 92±7% and after treatment was 86±3%. The total number of foACs and jbACs was reduced by 19±7% and 9±3%, respectively, with ACK treatment. RBC content was significantly reduced after treatment of both foACs (36±14% before and 14±3% after treatment) and jbACs (21±6% before and 7±2% after).

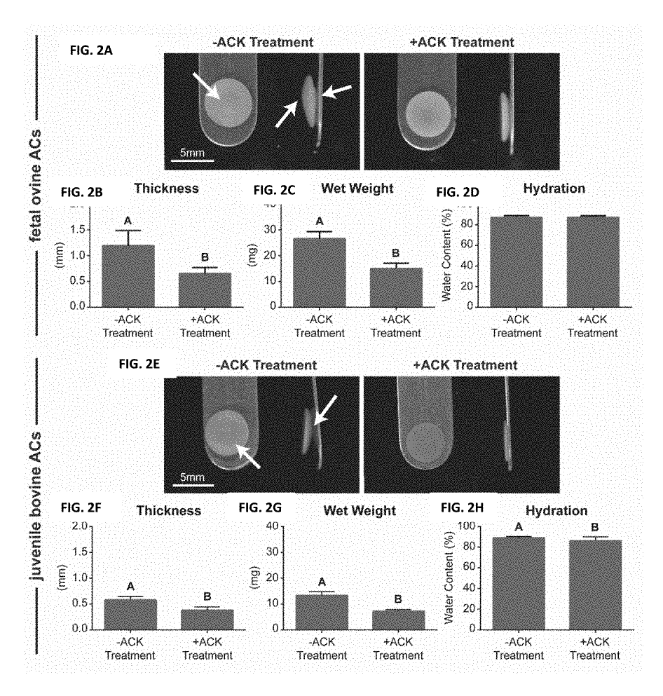

FIG. 2 shows the gross morphology of self-assembled neocartilage constructs after 6 weeks of culture. All constructs appeared hyaline-like with similar diameters. Bulbous, diffuse regions (indicating areas where “bad” cells could not make functional cartilage; these “bad” cells may have exhibited fragmented/inactive cytoskeleton, reduced membrane surface area, and/or altered cell stiffness) were present within both foAC and jbAC untreated groups. ACK treatment eliminated these regions and yielded flat foAC and jbAC neocartilage. ACK treatment also reduced the thickness and wet weight of both foAC and jbAC neocartilage constructs. Thickness of foAC neocartilage was 1.2±0.1 mm without treatment, and was significantly reduced to 0.7±0.1 mm with treatment. Thickness of jbAC neocartilage was 0.58±0.1 mm without treatment, and was significantly reduced to 0.38±0.1 mm with treatment. Wet weight of foAC neocartilage was 26.6±0.8 mg without treatment, and was significantly reduced to 15.1 ±0.6 mg with treatment. Wet weight of jbAC neocartilage without treatment was 13.3±0.4 mg, and was significantly reduced to 7.3±0.2 mg with treatment. Hydration of foAC neocartilage was 87.1±0.5% without ACK treatment and 87.2±0.4% with treatment. Hydration of jbAC neocartilage was 89.0±0.3% without ACK treatment, and was significantly reduced to 86.4±0.9% with treatment.

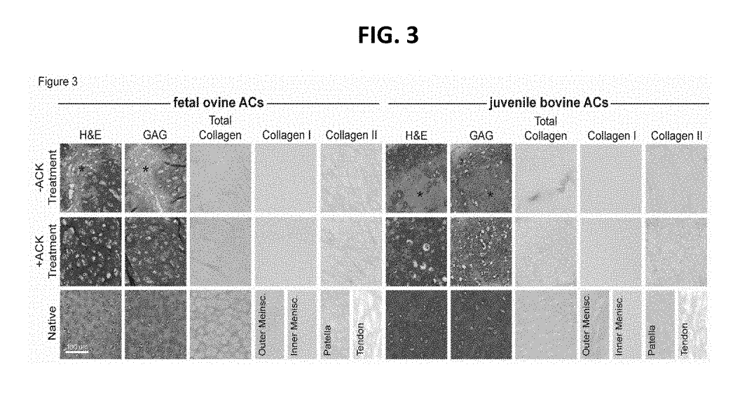

FIG. 3 shows neocartilage construct histology and immunohistochemistry after 6 weeks of culture. Histology showed the presence of diffuse, GAG-rich regions of low cellularity in both untreated foAC and jbAC neocartilage. ACK treatment eliminated these diffuse regions, yielding homogeneous tissue staining more intensely for GAG and collagen in both foAC and jbAC constructs. Intense GAG staining was present across all groups, which was further increased with ACK treatment for both foAC and jbAC constructs. Collagen staining was present across all groups, but was additionally enhanced by ACK treatment for both foAC and jbAC constructs. Collagen I staining was not preset in either the untreated or treated foAC and jbAC constructs. Collagen II staining was present in both untreated foAC and jbAC constructs and was intensified by ACK treatment.

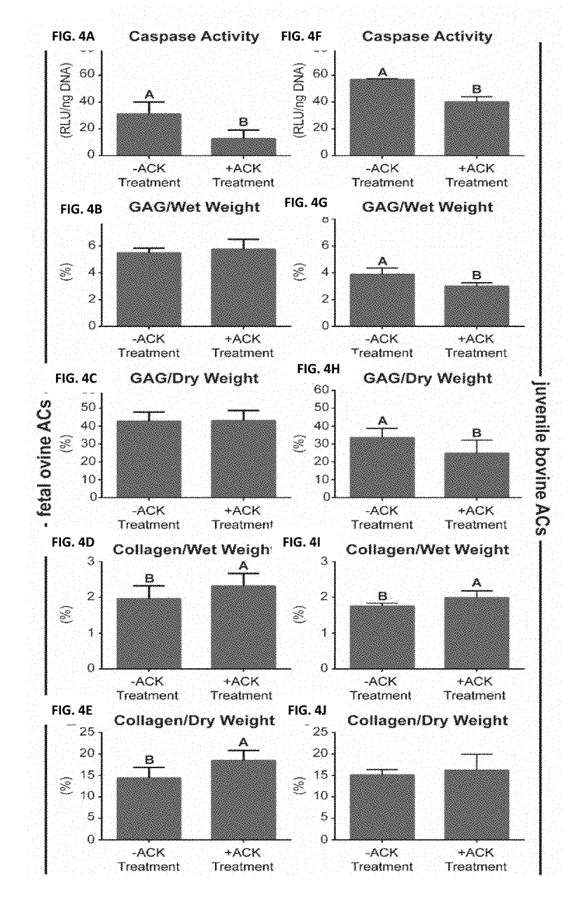

FIG. 4 demonstrates biochemical content of the neocartilage constructs. Untreated and ACK treated foAC neocartilage GAG per wet weight (GAG/WW) was 5.5±0.1% and 5.7±0.2%, respectively. Untreated and ACK treated foAC neocartilage GAG per dry weight (GAG/DW) was 42.8±1.5% and 43.1±1.7%, respectively. GAG per DNA in untreated foAC constructs was 60.4±0.9 pg/pg, and was significantly reduced to 50.54±1.3 pg/pg with ACK treatment. ACK treatment significantly decreased jbAC construct GAG per wet weight from 3.9±0.2% to 3.0±0.1° A and GAG per dry weight from 33.5±2.0% to 24.8±2.8%. ACK treatment significantly reduced jbAC construct GAG per DNA from 70.65±5.3 pg/pg to 28.1±1.4 pg/pg.

Collagen content per wet weight (collagen/WW) and collagen per dry weight (collagen/DW) in foAC neocartilage were significantly increased from 2.0±0.1% to 2.3±0.1% and 14.4±0.8% to 18.5±0.7%, respectively, by ACK treatment. Construct collagen per DNA in untreated and ACK treated foAC neocartilage was 20.5±0.9 pg/pg and 20.4±0.8 pg/pg, respectively. ACK treatment significantly increased collagen per wet weight from 1.8±0.1° A to 2.0±0.1° A in jbAC constructs. Collagen per dry weight in the untreated jbAC constructs was 15.2±0.5% and 16.3±1.4% in the ACK treated constructs. Collagen per DNA in untreated jbAC constructs was 31.7±1.2 pg/pg and was significantly reduced to 18.6±0.7 pg/pg with ACK treatment.

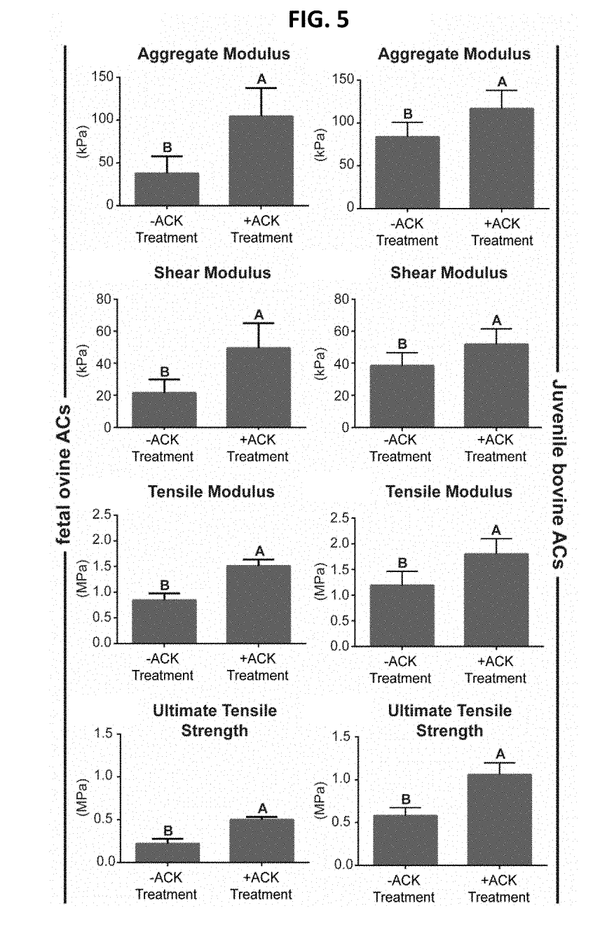

FIG. 5 shows mechanical properties of neocartilage constructs. ACK treatment significantly enhanced the compressive, shear, and tensile properties of both foAC and jbAC neocartilage constructs. Aggregate modulus of foAC constructs significantly increased from 37.8±8.1 kPa to 104.5±13.5 kPa with ACK treatment. ACK treatment similarly and significantly increased jbAC construct aggregate modulus from 83.8±7.0 kPa to 116.6±8.8 kPa. Shear moduli of foAC and jbAC neocartilage were significantly increased from 21.6±3.5 kPa to 49.4±6.4 kPa and 38.5±3.3 kPa to 51.9±4.0 kPa, respectively, by ACK treatment. ACK treatment significantly increased foAC construct tensile modulus from 0.8±0.1 MPa to 1.5±0.1 MPa and ultimate tensile strength (UTS) from 0.2±0.1 MPa to 0.5±0.1 MPa. Tensile modulus of jbAC constructs significantly increased from 1.2±0.1 MPa to 1.8±0.1 MPa, and UTS significantly increased from 0.6±0.1 MPa to 1.1±0.1 MPa as a result of ACK treatment.

### EXAMPLE 2

### Shearing

Example 2 describes methods of using shearing to select cells based on cytoskeletal properties. Example 2 shows a protocol by which to purify articular chondrocytes with the application of shear.

Cell isolation: Juvenile ovine articular chondrocytes (joACs) are to be isolated from the femoral condyles and trochlear groove of juvenile Rambouillet Suffolk cross sheep to be obtained from a local abbotoir (Nature's Bounty Farms, Dixon, Calif.) within the same day of animal sacrifice. Cartilage is to be minced into 1-2 mm³ cubes and washed two times with wash medium (Dubelco's Modified Eagle Medium; DMEM containing 1% (v/v) PSF). Minced cartilage is to be digested with 500 units/mL collagenase type 2 (Worthington Biochemical) in chondrogenic medium +3% (v/v) fetal bovine serum (FBS; Atlanta Biologicals) for 18 hours at 37° C. and 10% CO2 on an orbital shaker. Cells are then to be strained through a 70 μm strainer and counted.

Protocol for introducing shear to purify chondrocytes: (1) Place approximately 30 mL cell solution in conical tubes. (2) Attach the conical tube filter containing a mesh size of 15-20 μm such that the vacuum will force the flow of the cell solution into the new conical tube. (3) Attach the new conical tube to the opposing size of the vacuum filter and attach the filter to the vacuum line. (4) Invert the conical tube and filter set up so that the cell solution flows through the filter into the new conical tube. Wait until all solution has passed through the filter. (5) Detach the filter and old conical tube. Wash the filtered cell solution twice with wash medium and count the remaining cells.

### EXAMPLE 3

### Impact/Compression

Example 3 describes methods of using an impact/compression to select cells based on cytoskeletal properties. Example 3 shows a protocol by which to purify articular chondrocytes with the application of compression/impact.

Cell isolation: Juvenile ovine articular chondrocytes (joACs) are to be isolated from the patellofemoral surfaces of 1-year-old Rambouillet Suffolk cross sheep to be obtained from a local abattoir (Superior Farms, Dixon, Calif.) within 48 hours of slaughter (n=8). Cartilage from the surface of both condyles and the trochlear groove is to be minced into approximately 1 mm3 pieces and washed three times with Dulbecco's Modified Eagle Medium containing 4.5 g/L glucose and GlutaMAX (DMEM; Gibco) and 2% (v/v) penicillin/streptomycin/fungizone (PSF; Lonza). The cartilage is then to be digested in 0.2% (w/v) collagenase type II (Worthington) in DMEM containing 3% (v/v) fetal bovine serum (FBS; Atlanta Biologicals) for 18 hours at 37° C. with gentle rocking. After digestion, the resultant cell solutions are to be filtered through 70 μm cell strainers.

Protocol for introducing compression/impact to purify chondrocytes: (1) Place approximately 30 mL cell solution in conical tubes. (2) Add 5 glass beads of 0.5-1.25 mm diameter to the tubes. (3) Gently roll the conical tubes on plate rocker for 3 minutes. (4) Pipette the cell solution into ne conical tubes. Wash the glass beads with wash medium three times and place these wash solutions in the new conical tubes as well. (5) Wash the processed cell solution twice with wash medium and count the remaining cells.

B. Purification Based on Membrane Surface Area Properties

### EXAMPLE 4

### Hypotonic Solution

Example 4 describes methods of using a hypotonic solution to select cells based on membrane surface area properties. Example 4 shows that native-like neocartilage is achieved using multiple-passaged chondrocytes. The present invention is not limited to the methods or compositions described herein. In Example 4, the cartilage engineering model of the self-assembling process was used. Without wishing to limit the present invention to any theory or mechanism, it is believed that treatment of primary cartilage cells with a hypotonic buffer is effective at increasing viable chondrocyte purity by reducing the population of cells with pre-existing undesirable membrane surface area properties. It is believed then that this treatment produces a population of cells, enriched for viable chondrocytes without pre-existing undesirable membrane surface area characteristics, thereby increasing the functional properties of the resulting self-assembling neocartilage.

Example 4 shows that mimicking cell proliferation (chondrogenically tuned expansion), condensation, differentiation (aggregate redifferentiation culture), cartilaginous matrix production (self-assembly), and matrix maturation in vitro (using cartilage cells that were purified with a hypotonic solution and then extensively passaged) yields neocartilage with mechanical properties on par with native articular cartilage from which cells were sourced. Example 4 describes three phases. In Phase 1, seeding density was determined for both primary and passaged/redifferentiated chondrocytes, e.g., seeding density that yields neocartilage constructs with the greatest functional properties (and to select the culture system that requires the fewest number of chondrocytes). Without wishing to limit the present invention to any theory or mechanism, it is believed that under optimized culture conditions, mimicking the developmental sequence of chondrogenically tuned cell expansion, aggregation, and aggregate redifferentiation yields neocartilage from purified, multiple-passaged cells on par with neocartilage from primary cells. Phase 2 determined the utility of cytochalasin D and hyaluronidase treatments to further promote the chondrogenic redifferentiation of expanded chondrocytes. Without wishing to limit the present invention to any theory or mechanism, it is believed that a combinatorial treatment promotes cartilage-specific matrix production and increase neocartilage construct functional properties. Phase 3 promoted matrix formation and crosslinking-based maturation in neocartilage. Without wishing to limit the present invention to any theory or mechanism, it is believed that treatment with TGF-B1, c-ABC, and LOXL2 enhances the functional properties of neocartilage to be on par with native articular cartilage from which the cells were sourced.

Chondrocyte Isolation: Fetal ovine articular chondrocytes (foACs) were harvested from the patellofemoral surfaces of 120-day gestation Dorper cross sheep obtained as medical waste (UC Davis School of Veterinary Medicine). Cartilage from the whole surface of both condyles and the trochlear groove was minced into approximately 1 mm³ pieces, then washed and centrifuged (500 G for 5 minutes) three times with Dulbecco's Modified Eagle Medium containing 4.5 g/L glucose and GlutaMAX (DMEM; Gibco) and 2% (v/v) penicillin/streptomycin/fungizone (PSF; Lonza). The tissue was digested in 0.2% (w/v) collagenase type II (Worthington) in DMEM containing 3% (v/v) fetal bovine serum (FBS; Atlanta Biologicals) for 18 hours at 37° C. with gentle rocking. After digestion, the resultant cell solutions were filtered through 70 μm cell strainers. For Studies 1-3, foACs were washed with ACK buffer (154.4mM ammonium chloride (Sigma), 10mM potassium bicarbonate (Fisher Scientific), 50mM EDTA tetrasodium salt (Acros Organics) in ultrapure water for three minutes as previously described. These primary (P0) foACs were then frozen in DMEM with 20% (v/v) DMSO (Sigma) and 10% (v/v) FBS.

Chondrocyte Expansion and Redifferentiation: Previously frozen P0 foACs were seeded in T-225 flasks at 1.5×10⁴ cells/cm² and expanded in chemically defined chondrogenic medium (CHG medium) (DMEM containing 1% PSF, 1% ITS+premix (BD Biosciences), 1% non-essential amino acids (Gibco), 100 nM dexamethasone (Sigma), 50 mg/mL ascorbate-2-phosphaste (Sigma), 40 g/mL L-proline (Sigma), and 100 mg/mL sodium pyruvate (Sigma)) with 2% FBS and chondrogenically tuned TFP supplementation (1 ng/mL TGF-81, 5 ng/mL bFGF, 10 ng/mL PDGF; all from PeproTech). Media was exchanged every 2-3 days. At confluence, cells were lifted with 0.5% Trypsin-EDTA (Gibco) for 5 minutes followed by digestion of the cell layers with DMEM containing 0.2% collagenase type II and 2% FBS for approximately 1 hour at 37° C., triturating every 20 minutes. The resulting cell solution was filtered through a 70 μm cell strainer and reseeded into T-225 flasks to achieve three passages (P3). P3 foACs underwent aggregate redifferentiation (P3R) as previously described. Briefly, 750,000 cells/mL CHG medium containing TGB supplementation (10 ng/mL TGF-81, 100 ng/mL GDF-5, 100 ng/mL BMP-2; all from PeproTech) were cultured in 100 mm×20 mm petri dishes coated with 1% (w/v) molecular biology grade agarose (Thermo Fisher Scientific) made with phosphate buffered saline (PBS; Sigma) to create a non-adherent environment. Aggregate cultures were maintained on an orbital shaker at 60 rpm for the first 3 days and remained static for the remainder of the 14-day redifferentiation period. Media was exchanged every 2-3 days. At the end of the culture period, aggregates were digested with 0.5% Trypsin-EDTA for 20 minutes, followed by 0.2% collagenase in DMEM with 2% FBS for approximately 2 hours at 37° C., triturating every 20 minutes. Following dissociation of the aggregates, cells were filtered through a 70 μm cell strainer and counted.

Chondrocyte Actin Visualization: In Phase 2, to visualize the effects of cytochalasin D treatment on cytoskeletal-mediated chondrogenic redifferentiation, F-actin staining was performed on untreated P0 foACs and both cytochalasin D treated and untreated P3 and P3R foACs. Approximately 8×10³ cells/cm² were allowed to attach to glass slides for 1 hour in the presence of 2% FBS. Non-adherent cells were washed away with two exchanges of PBS, followed by the fixation of attached cells in 3.9% formaldehyde in PBS for 10 minutes. After another two washes with PBS, fixed cells were permeabilized with 0.1% Triton-X 100 (Sigma) in PBS for 5 minutes. Following two washes with PBS, cells were stained with CF594-conjugated phalloidin (Biotium; 1:200 dilution in PBS) for 30 minutes. Excess stain was washed away with two exchanges of PBS and the cells were counterstained with DAPI-containing Vectashield (Vector Laboratories) and coverslipped for visualization using a Texas Red fluorescent channel.

Neocartilage Construct Seeding and Culture: P0, P3, or P3R foACs were self-assembled into engineered neocartilage constructs in non-adherent agarose wells. A sterile stainless steel mold consisting of 5 mm diameter cylindrical posts was inserted into a 48-well plate, each well containing 1 mL molten 2% (w/v) agarose to create a single agarose well per plate well. After solidification of the agarose at room temperature, the mold was removed. Agarose wells were filled with CHG medium exchanged twice over the course of 5 days to ensure saturation of the agarose before seeding. For each phase, cells were seeded into 5 mm agarose wells in 100 μL CHG medium per well. In Phase 1, P0 or P3R foACs were each seeded at five densities: 2, 3, 4, 5, and 6 million cells per construct. In Phase 2, P3 and P3R foACs were seeded at 2 million cells per construct. In Phase 3, 2 million P3R foACs were seeded. All constructs were unconfined at day 6 and placed in larger wells coated with agarose to prevent construct adhesion. Media was exchanged daily prior to unconfinement and every other day after for the duration of the 6-week culture period. In Phase 1, no chemical treatments were applied during neocartilage culture. In Phase 2, cytochalasin D (Enzo Life Sciences; 2 μM at seeding and for the first 48 hours) and hyaluronidase (and 200 units/mL at seeding) were applied in a full-full factorial design. In Phase 3, cytochalasin D was applied as in the previous phase, as well as TCL treatment comprised of TGF-β1 (10 ng/mL throughout the entire culture duration), chondroitinase ABC (c-ABC, Sigma; 2 units/mL for 4 hours on day 7), and a LOX cocktail, applied days 7-21, consisting of lysyl oxidase-like 2 (LOXL2, Signal Chem; 0.15 pg/mL), copper sulfate (Sigma; 1.6 pg/mL), and hydroxylysine (Sigma; 0.146 μg/mL). For reference, P0 foACs that were not treated with ACK buffer during isolation (P0 Control) were also seeded at 4.5 million cells per construct and did not undergo other chemical treatments during neocartilage culture. All neocartilage evaluations were performed at the end of the culture period.

Neocartilage Gross Morphological Analysis: ImageJ (National Institutes of Health) was used to measure neocartilage construct diameter and thickness from pictures. Wet weights were obtained by weighing whole constructs before samples were portioned for histological, biochemical, and mechanical analysis.

Neocartilage Histological and Immunohistochemical Evaluation: Formalin-fixed samples were embedded in paraffin and sectioned along the short axis into 5 μm sections to expose the full thickness of the construct. In all studies, sections were stained with H&E to illustrate morphology, safranin O/fast green to show glycosaminoglycan (GAG) deposition, and picrosirius red to visualize collagen. Von Kossa and alizarin red staining were also performed to view mineralization. Immunohistochemistry (IHC) was performed to stain for collagen I (Abcam ab34710, dilution 1:250), collagen II (Abcam ab34712, 1:4000 dilution). In Phase 1, IHC was also performed to stain collagen VI (Abcam ab6588, dilution 1:250) and collagen X (Abcam ab49945, 1:200 dilution).

Neocartilage Biochemical Evaluation: Biochemical samples were weighed to measure wet weights, lyophilized, and weighed again to measure dry weights. Dried samples were digested in 125 μg/mL papain (Sigma-Aldrich), 5 mM N-Acetyl-L-Cysteine, 5 mM EDTA, 100 mM Phosphate Buffer at 65° C. for 18 hours. Glycosaminoglycan (GAG) content was measured by a Blyscan assay kit (Biocolor). Collagen content was measured by a modified colorimetric chloramine-T hydroxyproline assay using hydrochloric acid. Sircol collagen standard (Bicolor) was used to generate a standard curve. PicoGreen dsDNA reagent (Invitrogen) was used to measure DNA content. Neocartilage collagen and GAG contents were normalized to wet weight, dry weight, and DNA content. Pyridinoline crosslinks quantified by high-performance liquid chromatography (HPLC) using pyridinoline standards (Quidel) as previously described. Pyridinoline content was normalized to wet weight and collagen content.

Neocartilage Mechanical Evaluation: Creep indentation compressive testing was conducted on punches (3 mm in diameter) from each construct by applying a flat, porous indenter tip (0.8 mm diameter) using loads ranging from 0.45 to 2 g to achieve 10-15% strain. A semi-analytical, semi-numeric, linear biphasic model and finite element analysis were used to obtain the aggregate modulus and shear modulus from the experimental data. Tensile testing was conducted in accordance with ASTM standards (ASTM D3039). Constructs were punched into dog-bone shaped specimens with gauge lengths of 1.92 mm, and paper tabs glued to the tissue outside the gauge length. The paper tabs were gripped in a TestResources machine (TestResources Inc.), and pulled at 1% of the gauge length per second until sample failure. A stress-strain curve was generated from the experimental data and the sample cross-sectional area measured via ImageJ analysis. A least-squares fit of the linear region of the curve was used to obtain the tensile modulus, and the maximum stress yielded the ultimate tensile strength (UTS).

Functionality Index Evaluation: A modified functionality index (FI; Equation 1) was used to quantitatively evaluate the neocartilage engineered in all phases against native fetal and juvenile ovine articular cartilage and to select culture conditions to carry forward to each phase. Based structure-function relationships within cartilage, the importance of both compressive and tensile properties during joint loading, the importance of biochemical properties for tissue integration, and the contribution of crosslinking to mechanical integrity, all factors were equally weighted. In the functionality index, G represents GAG/WW (%), C represents total collagen/WW (%), P represents pyridinoline/collagen (nmol/mg), E^{c} represents (compressive) aggregate modulus, and E^{T} represents tensile modulus. Subscripts nat and eng represent native and engineered tissues, respectively. Constructs with inconsistent thicknesses and abnormal morphologies, such as tears, ruptures, or bulbous regions were deemed unsuitable and were excluded from functionality index assessments.

$$FI = \frac{1}{5} ( ( 1 - \frac{{G}_{nat} - {G}_{eng}}{{G}_{nat}} ) + ( 1 - \frac{{C}_{nat} - {C}_{eng}}{{C}_{nat}} ) + ( 1 - \frac{{P}_{nat} - {P}_{eng}}{{P}_{nat}} ) + ( 1 - \frac{{E}_{nat}^{C} - {E}_{eng}^{C}}{{E}_{nat}^{C}} ) + ( 1 - \frac{{E}_{nat}^{T} - {E}_{eng}^{T}}{{E}_{nat}^{T}} ) ) \qquad Equation 1$$

Statistical Analysis: In Phase 1, a two-way analysis of variance (ANOVA) followed by a Tukey's post hoc test in Prism 7 (GraphPad Software) was used to analyze the quantitative neocartilage properties and functionality indices of the different seeding densities across two passage conditions. In Phase 2, a one-way ANOVA followed by a Tukey's post hoc test was performed to analyze the quantitative neocartilage properties and functionality indices amongst different treatment groups. In Phase 3, a Student's t-test was performed to analyze the quantitative properties and functionality indices between treatment groups. A sample size of n=6 per group was used. All data are presented as means±standard deviations. Significance was determined by P<0.05 and is indicated in figures displaying quantitative results by marking statistically different groups with different symbols.

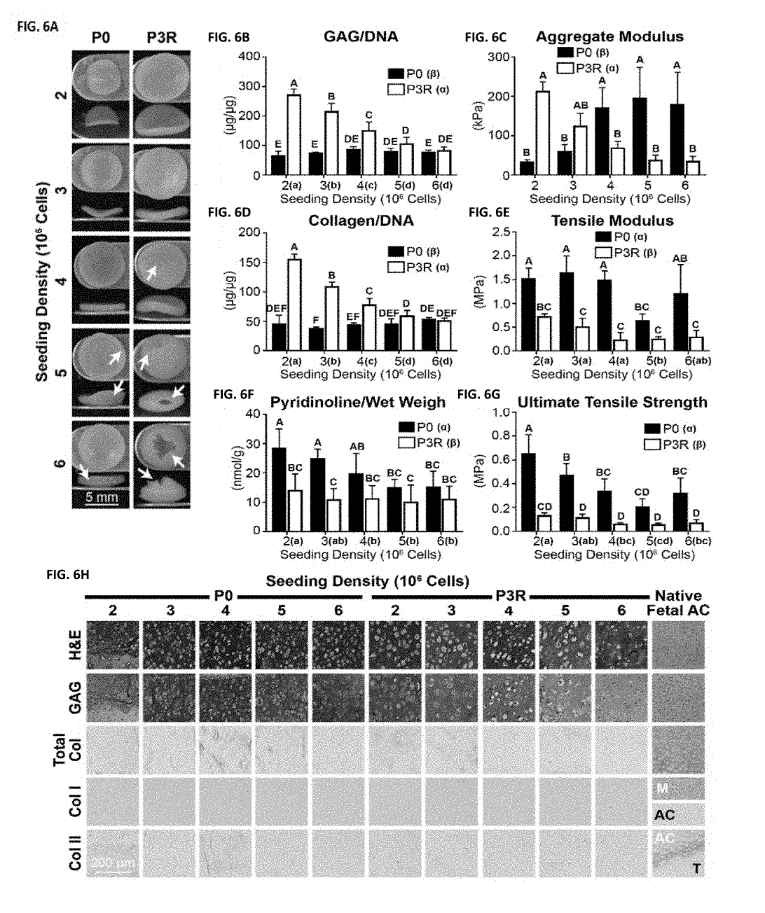

Phase 1: Neocartilage constructs showed dissimilarities in morphology based on passage and cell density (see FIG. 6). With respect to P0 neocartilage, construct diameter, thickness, and wet weight increased with greater cell seeding densities. The diameters of P0 constructs seeded at 2, 3, 4, 5, and 6 million cells, as well as the diameters of P3R constructs seeded at the same cell densities were 5.3±0.2 (FIG. 6E), 6.2±0.2 (FIG. 6D), 6.9±0.2 (FIG. 6C), 7.1±0.3 (FIG. 6C), 7.2±0.1 (FIG. 6C), 8.2±0.2 (FIG. 6A), 8.2±0.1 (FIG. 6A, FIG. 6B), 7.8±0.3 (FIG. 6B), 7.2±0.1 (FIG. 6C), and 7.0±0.2 (FIG. 6C) mm, respectively. The thicknesses of P0 constructs seeded at 2, 3, 4, 5, and 6 million cells, as well as the diameters of P3R constructs seeded at the same cell densities were 0.5±0.0 (FIG. 6F), 0.5±0.0 (FIG. 6F), 0.7±0.1 (FIG. 6E), 0.7±0.2 (FIG. 6D, FIG. 6E), 0.9±0.1 (FIG. 6B, FIG. 6C), 0.9±0.0 (FIG. 6B, FIG. 6C, FIG. 6D), 1.0±0.0 (FIG. 6B), 1.2±0.1 (FIG. 6A), 0.7±0.0 (FIG. 6C, FIG. 6D, FIG. 6E), 0.8±0.1 (FIG. 6B, FIG. 6C, FIG. 6D, FIG. 6E) mm, respectively. The wet weights of P0 constructs seeded at 2, 3, 4, 5, and 6 million cells, as well as the diameters of P3R constructs seeded at the same cell densities were 12.8±0.5 (FIG. 6G), 19.0±0.6 (FIG. 6F), 30.2±3.5 (FIG. 6E), 35.2±5.2 (FIG. 6D), 39.5±1.9 (FIG. 6D), 49.5±1.9 (FIG. 6C), 53.6±1.4 (FIG. 6B, FIG. 6C), 58.2±1.3 (FIG. 6A, FIG. 6B), 59.3±3.6 (FIG. 6A), 58.4±4.0 (FIG. 6A) mg, respectively. Constructs seeded at densities of 2, 3, and 4 million cells appear homogeneous, disc-shaped, and maintained a consistent thickness within each construct. Although of consistent thickness, constructs of 2 and 3 million cells were curved, while constructs of 4 million cells were flat. Constructs seeded at 5 and 6 million cells showed irregular morphologies including inconsistent thicknesses and folded and ruptured edges. In P3R neocartilage, generally, construct diameter decreased while thickness and wet weight increased with greater seeding density. Constructs seeded at 4 million cells displayed small, well-defined pockets of diffuse matrix of lower cellularity. At seeding densities of 5 and 6 million cells, these regions ruptured, causing the constructs to form two distinct layers, with only one layer fully intact. Reported thicknesses for these constructs were measured from the intact layer.

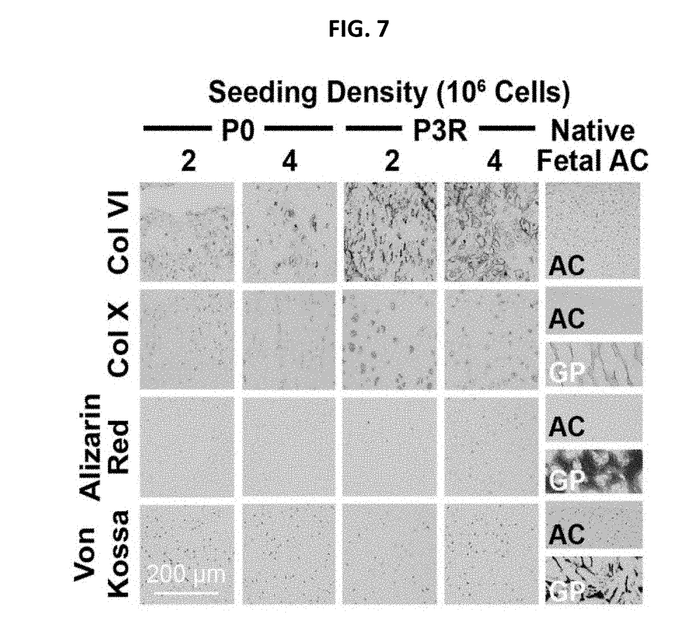

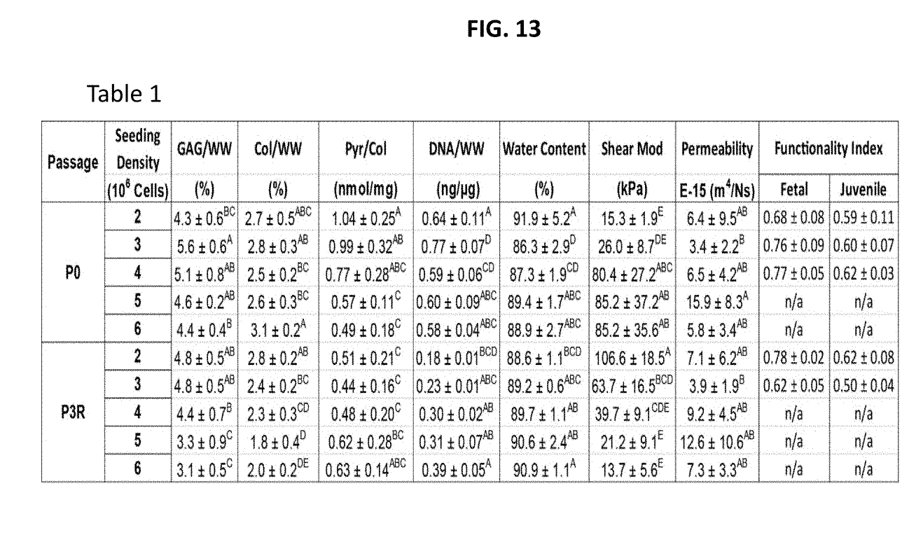

Histologically, differences in cell morphology and intensity of GAG, collagen, and collagen II staining, as a function of passage and neocartilage seeding density, were observed (see FIG. 6). H&E staining revealed larger chondrocytes in both the P0 and P3R constructs than those present in native tissue. Additionally, the lacunae surrounding the cells in P3R constructs were larger than those in P0 neocartilage. Safranin O staining for sulfated GAGs showed more intense staining in both the P0 and P3R constructs compared to native tissue. Safranin O stained less intensely in the outer regions of the P0 constructs as compared to the central region. This outer region was greatly reduced in the P3R constructs. GAG staining appeared most intense at a seeding density of 4 million cells in P0 neocartilage. In P3R neocartilage, GAG staining was most intense at the 2 million cell density and decreased with increasing seeding density. Picrosirius red staining for collagen was less intense in both the P0 and P3R neotissues compared to native tissue. The outer region of the P0 and P3R constructs stained more intensely than the inner regions, and these regions were thinner in the P3R neocartilage. Picrosirius red staining was most intense at a seeding density of 4 million cells in P0 neocartilage and 2 million cells in P3R neocartilage. Collagen I staining was minimal across all groups. Within P0 neocartilage, collagen II staining peaked at a seeding density of 4 million cells. Within P3R neocartilage, collagen II staining was most intense at the seeding density of 2 million cells and decreased with increasing seeding density. Additional staining for collagen VI, collagen X, alizarin red, and von Kossa are shown in FIG. 7. Both P0 and P3R neocartilage stained positively for collagen VI, with P3R neocartilage staining the darkest. P0 and P3R neocartilage also stained faintly for collagen X within the lacunae but not the surrounding ECM of the neocartilage. Neocartilage of all passages and seeding densities did not stain with alizarin red or Von Kossa. Biochemical contents, mechanical properties, and functionality index calculations are listed in Table 1 of FIG. 13 and shown in FIG. 6. The functionality indices identified the optimal P0 (P0 Opt) and P3R (P3R Opt) seeding densities as 4 million and 2 million cells/construct, respectively. Based on a superior functionality index, the P3R group seeded at 2 million cells/construct was moved forward to Phase 2.

For reference, neocartilage grown from P0 foACs that were not treated with ACK buffer at isolation were also mechanically tested. These constructs were seeded at a density of 4.5 million cells/construct based on methods in previous studies with P0 foACs. The aggregate modulus, shear modulus, and permeability were 97.7±20.4 kPa, 43.1±12.1 kPa, 45.1±15.7×10¹⁵ m⁴/Ns, respectively. The tensile modulus and UTS were 0.8±0.2 MPa and 0.2±0.1 MPa, respectively.

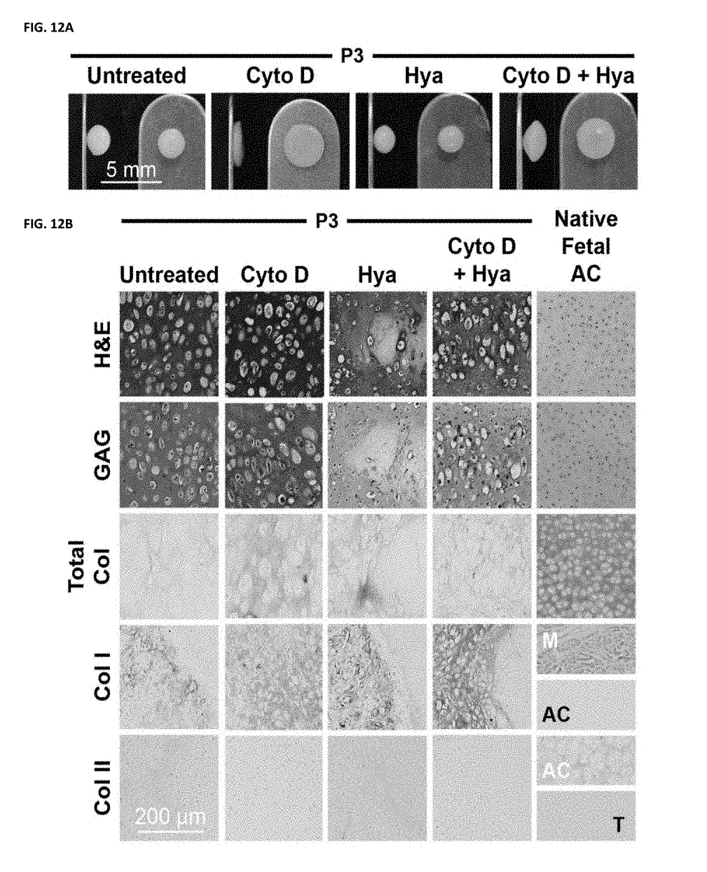

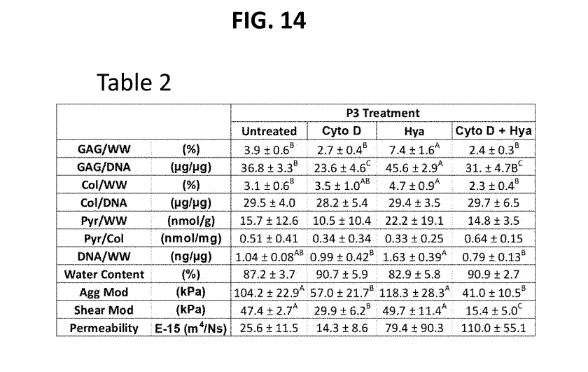

Phase 2: In the first study of this phase, cytochalasin D and hyaluronidase were examined to determine if a chemical treatment was capable of redifferentiating passaged chondrocytes without using aggregate redifferentiation. P3 neocartilage constructs showed great morphological differences from P3R neocartilage. In P3 neocartilage (see FIG. 12), cytochalasin D (Cyto D) treatment resulted in the only flat and homogeneous construct. No treatment (Untreated), hyaluronidase treatment (Hya), or dual treatment (Hya +Cyto D) resulted in rounded neocartilage with a diffuse void space in the center of the construct. The diameters of untreated, cytochalasin D treated, and dual treated constructs were 2.8±0.2, 3.7±0.2, 2.6±0.1, and 3.4±0.2 mm, respectively. The diameter of the cytochalasin D treated neocartilage was significantly greater than those of the untreated, hyaluronidase treated, and dual treated neocartilage. The diameter of the dual treated neocartilage was also significantly greater than that of the untreated and hyaluronidase treated neocartilage. The thicknesses of neocartilage resulting from no treatment, hyaluronidase treatment, or the dual treatment were 2.1±0.2, 0.4±0.1, 2.0±0.2, and 1.5±0.7 mm, respectively. The cytochalasin D treated neocartilage was significantly thinner than the neocartilage of the other groups. The wet weights of untreated, cytochalasin D treated, and dual treated neocartilage were 7.1±0.6, 8.4±1.0, 6.7±0.4, and 10.1±1.3 mg, respectively. The wet weight of the dual treated group was significantly greatest above the other groups. The wet weight of the cytochalasin treated group was also significantly greater than those of the untreated and hyaluronidase treated groups. Histologically, void regions were present in the untreated, hyaluronidase treated, and dual treated groups (see FIG. 12). All treatments, except for hyaluronidase, resulted in darker staining than native fetal ovine articular cartilage. Total collagen staining for all groups was less intense than staining for the native control. Cytochalasin D treatment resulted in the strongest GAG and total collagen staining. All constructs stained for collagen I on par with native fetal ovine meniscus, and did so particularly intensely in the untreated, hyaluronidase treated, and dual treated neocartilage around the inner diffuse region. All constructs stained minimally for collagen II. P3 biochemical and mechanical data are shown in Table 2 of FIG. 14. Cytochalasin D and hyaluronidase treatments without aggregate redifferentiation were incapable of decreasing collagen I production and increasing collagen II production in P3 neocartilage.

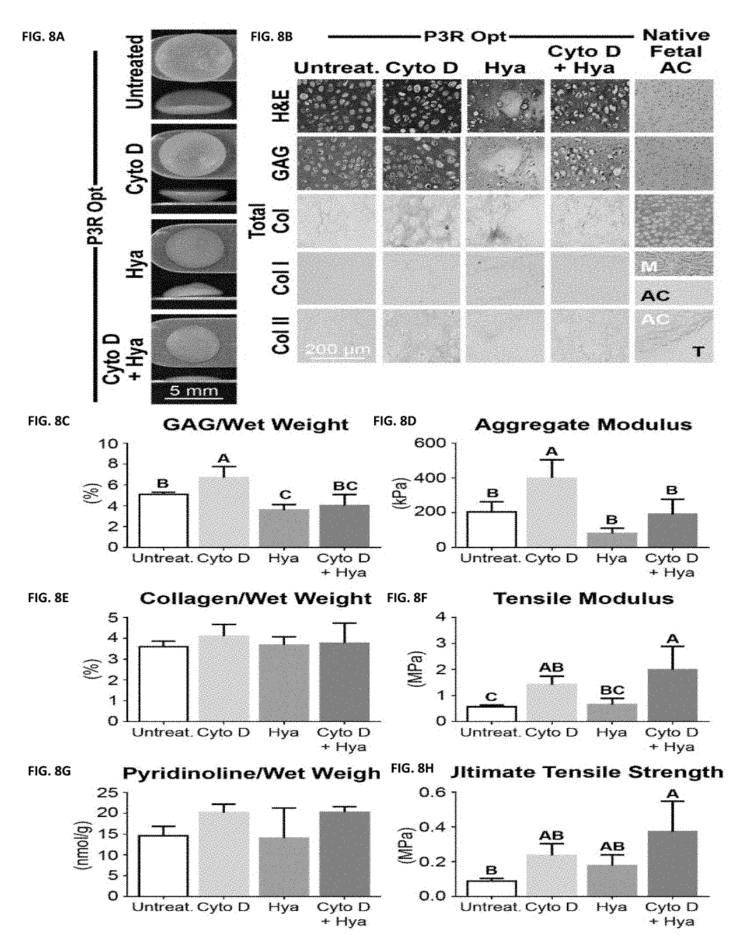

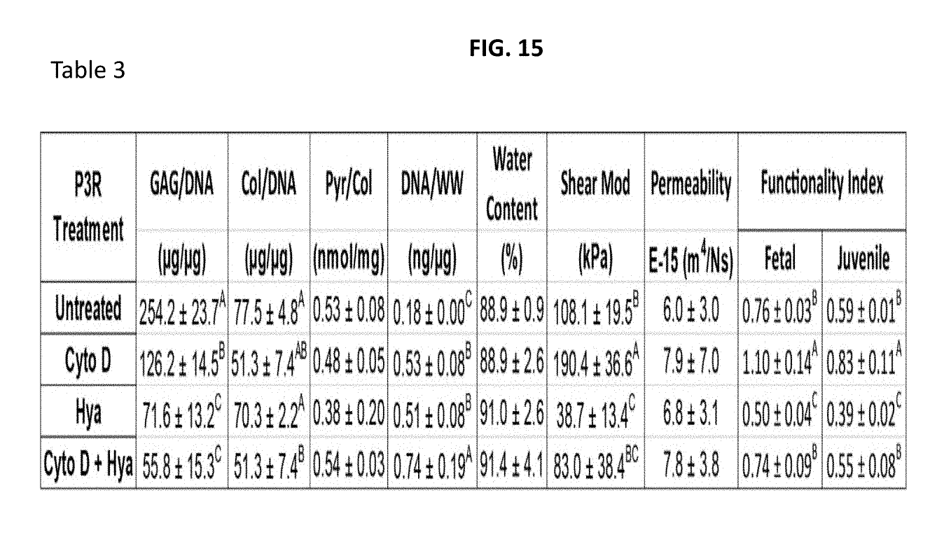

In the second study of this phase, aggregate redifferentiation was introduced in conjunction with cytochalasin D and hyaluronidase treatment of P3R Opt neocartilage carried forward from Phase 1. In P3R neocartilage (see FIG. 8), P3R Opt, cytochalasin D treatment (Cyto D), and dual treatment (Cyto D +Hya) resulted in constructs of uniform thickness. Hyaluronidase treatment (Hya) resulted in the formation of a diffuse void region in the center of the constructs. All constructs, except for the dual treated group, were slightly bowl-shaped. The diameters of untreated, cytochalasin D treated, and dual treated constructs were 8.2±0.2, 6.5±0.2, 5.9±0.0, and 5.7±0.3 mm, respectively. The construct diameter of the untreated group was significantly greater than those of the other treatment groups. The construct diameter of the cytochalasin D treated group was significantly greater than those of the hyaluronidase and dual treated groups. The thicknesses of neocartilage resulting from no treatment, cytochalasin D treatment, hyaluronidase treatment, and the dual treatment were 0.9±0.0, 0.7±0.1, 0.8 ±0.2, and 0.4±0.1 mm, respectively. The thickness of dual treated neocartilage was significantly less than those of the other treatment groups. The wet weights of untreated, cytochalasin D treated, and dual treated neocartilage were 50.1±3.2, 28.1±2.6, 23.9±1.4, and 11.8±3.9 mg, respectively. Histologically, a diffuse void region was present in only the hyaluronidase treated group (see FIG. 8). GAG, total collagen, and collagen II staining was most intense in the cytochalasin D treated neocartilage. Biochemical and mechanical data, as well as functionality indices are shown in FIG. 8 and FIG. 15 (Table 3). Based on a superior functionality index, cytochalasin D treatment was selected to move forward to Phase 3.

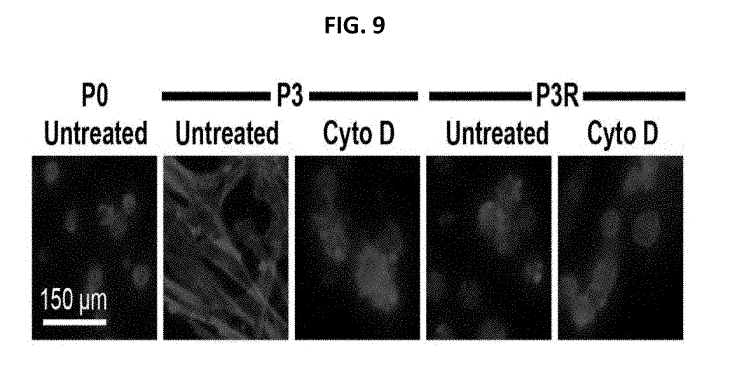

Fluorescent staining of F-actin within chondrocytes showed marked differences between cell passage and treatment (see FIG. 9). In P0 chondrocytes, actin arrangement was cortical, manifesting as rings around the periphery of each cell. Untreated P3 chondrocytes were much larger in size and showed fibrillar actin arrangement within fibroblast-like cells. Cytochalasin D treatment of P3 chondrocytes induced a rounded cell shape and, while actin was still present throughout the cell, much of it localized to the perimeter. Untreated P3R chondrocytes showed cortical actin arrangement with some small fibrillar areas. Cytochalasin D treatment of P3R chondrocytes localized more of the actin cortically than in untreated P3R cells.

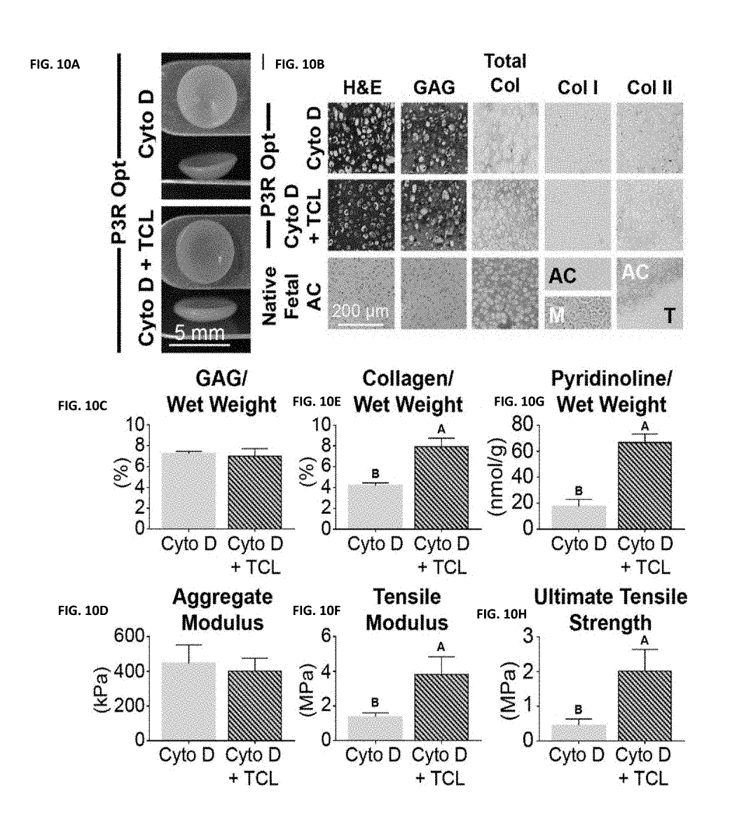

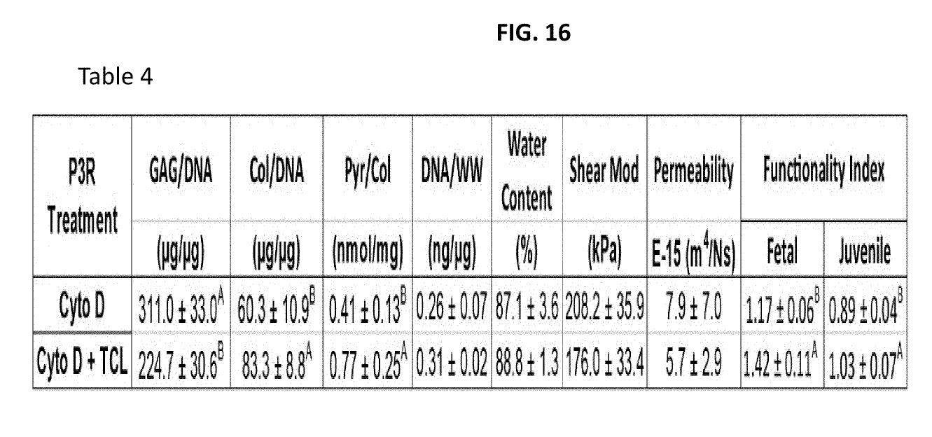

Phase 3: Having selected P3R Opt as the optimal group from Phase 1 and cytochalasin D treatment of P3R Opt neocartilage from Phase 2, Phase 3 examined the additional effect of TCL treatment on cytochalasin D treated, P3R Opt neocartilage. Neocartilage treated with both cytochalasin D and TCL appeared similar in shape and thicker than cytochalasin D treated neocartilage. The diameters of cytochalasin D treated and the dual cytochalasin D and TCL treated neocartilage were 6.5±0.4 and 6.4±0.3 mm, respectively. The thickness of the dual treated neocartilage was significantly greater than that of the cytochalasin D treated neocartilage: 1.1±0.1 and 0.8±0.2 mm, respectively. The wet weights of cytochalasin D treated and the dual treated neocartilage were 87.1±3.6 and 88.8±1.3 mg, respectively. Histologically, the neocartilage of both groups appeared homogeneous (see FIG. 10). Both groups stained more intensely for GAG and less intensely for total collagen than native fetal articular cartilage. The dual treated group stained more intensely for GAG, total collagen, and collagen II. Neither group stained for collagen I. Biochemical and mechanical data, as well as functionality indices are shown in FIG. 10 and FIG. 16 (Table 4). Functionality indices indicated that neocartilage treated with both cytochalasin D and TCL was superior to neocartilage treated with cytochalasin D alone.

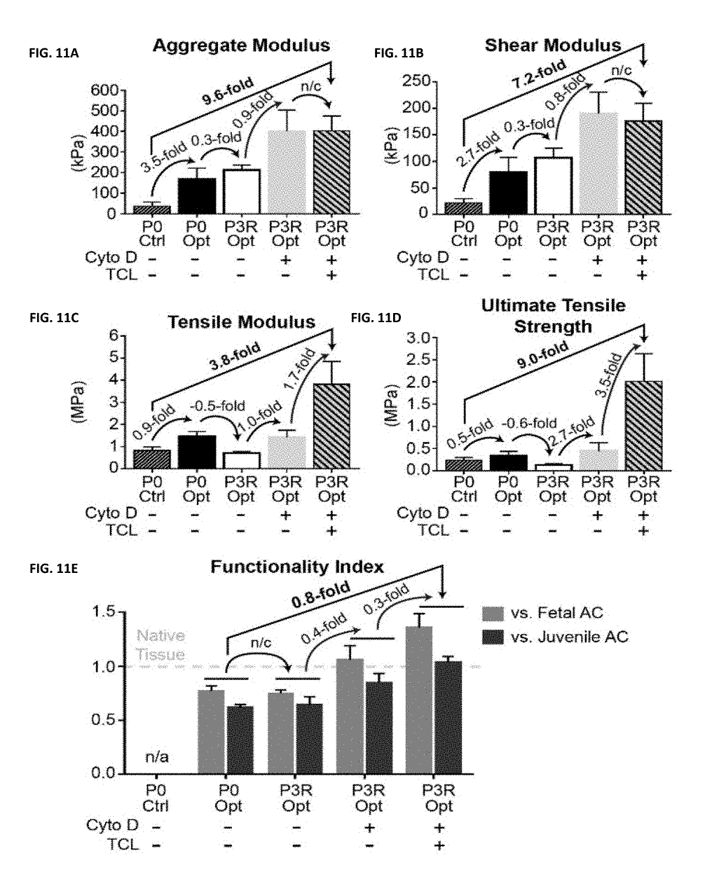

Example 4 describes how mimicking key salient aspects of tissue formation in vitro using purified and subsequently highly-passaged cells yielded neocartilage with mechanical properties on par with native articular cartilage from which cells were sourced. The progressive development of neocartilage functionality through Phases 1-3 is shown in FIG. 11, demonstrating large increases in mechanical properties. Specifically, the neocartilage aggregate modulus, shear modulus, and tensile modulus were found to increase 9.6-fold, 7.2-fold, and 3.8-fold over P0 controls, while the tensile strength increased 9.0-fold. The neocartilage resulting from these successive studies achieved an FI of 1.42 when compared to native fetal cartilage and an FI of 1.03 when compared to native juvenile cartilage. This indicates that the engineered neocartilage exceeded native tissue values for the parameters measured by the functionality index, indicating that it is possible to achieve adult level properties.

In Phase 1, neocartilage from P3R cells could achieve P0 neocartilage properties. The functionality index of the P3R neocartilage seeded at the optimal density was on par with that of P0 neocartilage seeded at the optimal density. With respect to fetal ovine articular cartilage, P0 Opt achieved an FI of 0.77 and P3R Opt achieved an FI of 0.78. The use of multiple passaged cells to engineer functionally robust tissue has great translational impact, because it indicates that fewer cells may be isolated to engineer superior neocartilage. Thus, P3R Opt neocartilage was carried forward to the subsequent phases. In Phase 2, it was shown that only cytochalasin D treatment was required to produce superior neocartilage from passaged/redifferentiated cells. For example, cytochalasin D treatment of P3R Opt neocartilage resulted in a 0.9-fold increase in the compressive stiffness, a 1.0-fold increase in tensile stiffness, and a 2.7-fold increase in tensile strength, yielding an FI of 1.1 with respect to native fetal cartilage and 0.83 with respect to native juvenile cartilage. Thus, cytochalasin D treatment of P3R Opt neocartilage was carried forward. In Phase 3, the addition of TCL treatment was shown to promote crosslinking-based maturation and enhance neocartilage functional properties achieving an FI of 1.42 with respect to native fetal cartilage and 1.03 with respect to native juvenile cartilage. The aggregate modulus exceeded that of native fetal cartilage, and the tensile modulus was within range of native levels. This work represents a significant step toward achieving biomimetic articular cartilage and using multiple-passaged cells to do so.

In Phase 1, P3R Opt neocartilage achieved an FI on par with P0 Opt neocartilage. Within P0 neocartilage, construct functional properties increased with increasing seeding density until a plateau was reached. However, with P3R neocartilage, functional properties decreased with increasing seeding density. This is in contrast to traditional tissue engineering strategies that assume primary cells are more synthetically capable than passaged cells, and that great cell numbers are required to produce superior neotissues. In this study, chondrocytes were expanded over 4,000 times and seeded at a lower density than is required with primary cells to achieve neocartilage that is larger in diameter and with an equivalent FI. The aggregate modulus, GAG/DNA production, and collagen/DNA production of P3R Opt neocartilage were 0.3-fold, 2.2-fold, and 2.6-fold greater than those of P0 Opt neocartilage (see FIG. 6). Although collagen content in P3R Opt neocartilage was on par with that of P0 Opt neocartilage, the tensile stiffness and strength of P3R Opt neocartilage were greatly reduced. Given the importance of collagen crosslinks to the tensile properties of cartilage, the reduced pyridinoline content in P3R Opt neocartilage was likely the reason for this, as addressed with TCL treatment in Phase 3. By using chondrogenically tuned expansion and aggregate redifferentiation methods, as well as optimizing self-assembling culture conditions, it was possible to engineer robust neocartilage from multiply passaged cells; achieving this required 8,000 times fewer primary cells than engineering neocartilage from non-passaged cells.

By examining multiple seeding densities across passage conditions, it was unexpected that passaged chondrocytes can be recalibrated to exhibit more immature behaviors. At seeding densities of 2, 3, and 4 million cells, P3R chondrocytes were more synthetic than P0 chondrocytes. Example 4 mimicked the proliferation, condensation, differentiation, and tissue formation that occurs developmentally with in vitro steps, such as monolayer expansion, aggregate redifferentiation, and self-assembly. Evidence suggests that doing so recalibrated the P3R chondrocytes to a more immature state, which enabled the increased production of matrix molecules. For example, the matrix secreted by P3R chondrocytes better reflected the composition of articular cartilage ECM at early stages. As native cartilage matures, collagen VI staining that is present throughout the ECM localizes to the pericellular matrix and collagen II staining increases. Pyridinoline content within native cartilage also increases greatly over long time scales as cartilage matures. In this study, P3R neocartilage exhibited less intense collagen II staining, more intense collagen VI staining, and lower levels of pyridinoline compared to P0 neocartilage (see FIG. 6 and FIG. 7). These data support the assertion that P3R chondrocytes are at a more immature state than P0 chondrocytes.

A culture technique termed macromolecular crowding has been used to enhance cartilage matrix production and maturation by chondrocytes in monolayer, but shows negative effects in 3D culture. When Ficoll 70 and Ficoll 40 were applied to chondrocytes in monolayer, collagen II expression increased, as well as the production of GAG and total collagen. However, in a 3D pellet culture model, macromolecular crowding treatment led to cartilage matrix deterioration as early as day 2 in culture. The proinflammatory cytokine IL-6 was also detected in the medium of high density cultures, but not low density cultures. The present study showed that P3R chondrocytes have the potential to be highly synthetic. Matrix deposition in self-assembling neocartilage is known to begin as early as day 1 after cell seeding.

Passaged chondrocytes exhibit a strongly chondrogenic phenotype in self-assembled neocartilage. With the use of redifferentiated chondrocytes and the newly proposed mechanism of self-induced macromolecular crowding and inflammatory cytokine-regulated matrix production, phenotypic verification of the chondrocytes in culture is necessary. In osteoarthritis, chondrocytes exhibit proliferation, increased synthesis of matrix molecules, including collagen X, hypertrophy, and mineralization. To verify the chondrogenic phenotype of P3R neocartilage, it and P0 neocartilage were stained for collagen VI, collagen X, alizarin red, and von Kossa (see FIG. 7). P3R neocartilage stained more intensely for collagen VI than P0 neocartilage. All groups stained faintly within the lacunae for collagen X, indicating that its presence was not caused by in vitro manipulations, such as chondrotuned expansion and aggregate redifferentiation, performed in this study. None of the groups stained with alizarin red or von Kossa, indicating there was no mineralization. While presence of collagen X, in addition to the large lacunae observed in P3R neocartilage may indicate a degenerative tissue or hypertrophic chondrocyte phenotype, no mineralization was present. Additionally, lacunae are reduced in size in high density P3R neocartilage. As collagen VI, collagen X, and large lacunae are signs of immature cartilage, these data further support that the recalibration of passaged chondrocytes to an immature phenotype has been achieved.

In Phase 2, cytochalasin D treatment of passaged/redifferentiated cells further enhanced their chondrogenic phenotype. Cytochalasin D treatment of P3R Opt neocartilage (carried forward from Phase 1) increased the aggregate modulus 0.9-fold over the untreated group (see FIG. 8) and to the level of native articular cartilage of adult sheep. Tensile stiffness and strength were also increased 1.0-fold and 2.7-fold, respectively, potentially due to minor concomitant increases in collagen and pyridinoline contents. This treatment yielded neocartilage with an FI of 1.1 with respect to native fetal cartilage and 0.83 with respect to native juvenile cartilage. The success of this treatment on passaged/redifferentiated cells motivated the study of its effect on passaged, non-redifferentiated (P3) chondrocytes. Cytochalasin D treatment of P3 neocartilage resulted in the only flat, homogeneous constructs of all treatments (see FIG. 12). However, its action alone was not enough to affect redifferentiation to a degree sufficient enough to manifest changes in the functional properties of the constructs, as illustrated by intense collagen I staining and low biochemical content and mechanical properties (see FIG. 14 (Table 2)). Functionality indices were not calculated for P3 neocartilage of any treatment because the constructs were not testable in tension and were morphologically unacceptable. Actin within P3R and P3 chondrocytes was visualized to confirm the method of action of cytochalasin D (see FIG. 9), similar to what is observed with P0 cells. Cytochalasin D treatment greatly improved the cortical organization of F-actin in P3 and P3R chondrocytes.

Phase 3 mimicked the progression of tissue formation by enhancing neocartilage matrix deposition and crosslinking to achieve native-level tensile properties. In Phase 1, pyridinoline/WW was greatly reduced in P3R neocartilage compared to P0 neocartilage. These levels remained consistently low in Phase 2. While this prevents neocartilage from achieving improved tensile properties, the late development of pyridinoline crosslinks compared to other matrix components mimics native cartilage maturation. TCL treatment, which has been shown to increase collagen content and crosslinking within the collagen network was applied in Phase 3. This treatment indeed resulted in a 0.9-fold increase in collagen/WW and a 2.9-fold increase in pyridinoline/WW, as well as a 1.7-fold increase in tensile stiffness and a 3.5-fold increase in tensile strength, without altering compressive stiffness. These tensile properties are within the range of what has been reported for juvenile sheep articular cartilage. TCL-treated neocartilage also achieved an FI of 1.42 with respect to native fetal cartilage and 1.03 with respect to native juvenile cartilage. This indicates that the properties of the neocartilage engineered in this work are now approaching adult levels. Mimicking key steps in native cartilage formation and following a developmentally inspired order of matrix development and maturation enabled purified, passaged/redifferentiated cell neocartilage to achieve tensile properties in the range of native cartilage.

By mimicking key aspects of native cartilage formation and applying developmentally inspired chemical stimuli, this work was able to engineer neocartilage from cells that had been expanded over 4,000 times with functional properties that approached native adult cartilage. Treatment with ACK lysing buffer, cytochalasin D, and TCL in addition to chondrogenically tuned expansion, aggregate redifferentiation, and optimized self-assembly of neocartilage yielded mature neocartilage with the greatest functionality index reported by our group. Additionally, it was shown that passaged cells may be recalibrated to a more synthetic state. A mechanism based on self-induced macromolecular crowding and cytokine-regulated feedback inhibition of cartilage matrix synthesis in high density 3D cultures provides a plausible explanation of seeding density-dependent matrix synthesis. Finally, an updated functionality index that accounted for the importance of tissue crosslinking was provided. This work makes strides toward establishing protocols to create native-like engineered neocartilage from 8,000 times fewer primary cells than previous methods.

### EXAMPLE 5

### Shearing

Example 5 describes methods of using a shear to select cells based on membrane properties. Example 5 shows a protocol by which to purify articular chondrocytes with the application of shear.

Cell isolation: Fetal sheep ACs are to be isolated from the femoral condyle and trochlear groove of the knees of Dorper cross sheep in 120-125 day gestation (UC Davis School of Veterinary Medicine). Minced cartilage tissue is to be washed with PBS and digested with 500 units/mL collagenase type 2 (Worthington Biochemical, Lakewood, N.J.) in chondrogenic medium +3% (v/v) FBS (Atlanta Biologicals, Lawrenceville, Ga.) for 18 h at 37° C/10% CO2. Cells are then to be strained through a 70 μm filter, washed with wash medium, and counted.

Protocol for introducing shear to purify chondrocytes: (1) Place approximately 50 mL cell solution in petri dishes. (2) Submerge the paddle rotor into the petri dish and rotate it at 20 rpm for 3 minutes. (3) remove the paddle rotor and wash the processed cell solution twice with wash medium and count the remaining cells.

### EXAMPLE 6

### Impact/Compression

Example 6 describes methods of using an impact/compression to select cells based on membrane properties. Example 6 shows a protocol by which to purify articular chondrocytes with the application of compression/impact.

Cell isolation: To obtain costal chondrocytes, cartilage from juvenile bovine stifle joints is to be minced into 1-2 mm³ pieces and digested in 0.2% type II collagenase (Worthington) in Dulbecco's modified Eagle's medium (DMEM) (Gibco) with 1% penicillin/streptomycin/fungizone (PSF) (BD Biosciences) and 3% fetal bovine serum (Atlanta Biologicals) for 18 hours at 37° C. After digestion, chondrocytes are to be filtered through 70 μm cell strainers, resuspended in blank DMEM, and counted.

Protocol for introducing compression/impact to purify chondrocytes: (1) Place approximately 20 mL cell solution in conical tubes. (2) Centrifuge the cell solution at 300 g for 5 minutes such that a pellet forms. (3) Insert the associated mesh conical pestle. Ensure the mesh size is smaller than 15 μm. Gently compress the cell pellet with the mesh pestle once every 30 seconds for 3 minutes. (4) Remove the pestle and wash the processed cell solution twice with wash medium and count the remaining cells.

C. Purification based on Stiffness Properties

### EXAMPLE 7

### Hypotonic Solution

Example 7 describes methods of using a hypotonic solution to select cells based on stiffness properties. Without wishing to limit this invention to any particular theory or mechanism, it is believed that treatment of cartilage cells with a hypotonic buffer is effective to increase viable chondrocyte purity by reducing the population of cells with a pre-existing undesirable stiffness characteristics. Therefore, this treatment produces a population of cells, enriched for viable chondrocytes without undesirable stiffness characteristics, thereby increasing the functional properties of the resulting self-assembling neocartilage.

Example 7 shows that native cartilage compressive properties are achieved in engineered neocartilage. The present invention is not limited to the methods or compositions described herein.

In Example 7, chondrocytes were isolated from the stifle joints of fetal sheep, a highly clinically-translatable cell source. Firstly, the treatment of primary (P0) fetal chondrocytes with ammonium-chloride-potassium lysing buffer (ACK buffer) was examined to determine its effects on chondrocyte purity within the cell isolate and resulting self-assembling neocartilage functional properties. Chondrocyte purity was evaluated by cell counting. Neocartilage functional properties were evaluated with a standard battery of assays, including compressive creep indentation, uniaxial tensile testing, GAG, collagen, and DNA assays, as well as histology and IHC. Secondly, the seeding density of P0 and passaged, redifferentiated (P3R) fetal chondrocytes during the self-assembling process was examined. Cells were seeded at 2, 3, 4, 5, and 6 million cells per 5 mm construct, and the same set of assays were used to evaluate the resulting neocartilage functional properties. Lastly, cytochalasin D and hyaluronidase were applied at the beginning of the self-assembling process in a full-factorial design to examine their ability to further enhance the resulting neocartilage functional properties. Neocartilage was evaluated with the standard battery of assays.

Results: ACK buffer treatment of freshly isolated P0 fetal chondrocytes decreased red blood cell contamination in the cell isolate by 60%. ACK treatment significantly increased neocartilage 1) aggregate modulus by 1.8-fold, 2) shear modulus by 1.3-fold, and 3) tensile modulus by 0.8-fold. Carrying forward ACK treatment of chondrocytes during isolation, the seeding density of P0 chondrocytes was optimized to 4 million cells/construct, additionally increasing neocartilage aggregate modulus by 0.6-fold and shear modulus by 0.8-fold. After passaging and redifferentiation (P3R) of these cells, seeding density was then optimized to 2 million cells/construct, further increasing aggregate modulus by 0.3-fold and shear modulus by 0.3-fold. Cytochalasin D application during the self-assembling of ACK treated P3R chondrocytes seeded at 2 million cells/construct significantly increased neocartilage aggregate modulus to 400 kPa, 9.6-fold over the P0 control.

As previously described, the present invention features methods for engineering cartilage with compressive properties generally akin to native cartilage. The method features purifying isolated chondrocytes (e.g., via hypotonic lysis buffer), optimizing neocartilage seeding density, re-differentiating passaged chondrocytes via novel aggregate culture methods such that primary cell neocartilage properties are preserved, and/or enhancing chondrocyte activity via cytoskeleton-modifying agents.

### EXAMPLE 8

### Shearing

Example 8 describes methods of using a shearing to select cells based on stiffness properties. Example 8 shows a protocol by which to purify articular chondrocytes with the application of shear. Cell isolation: Fetal ovine articular chondrocytes (foACs) are to be isolated from the stifle joints of 120-day gestation Dorper cross sheep. Cartilage from the condyles and the trochlear groove is to be minced into approximately 1 mm³ pieces, washed and centrifuged (500 G for 5 minutes) three times with Dulbecco's Modified Eagle Medium containing 4.5 g/L glucose and GlutaMAX (DMEM; Gibco) and 2% (v/v) penicillin/streptomycin/fungizone (PSF; Lonza). The tissue is to be digested in 0.2% (w/v) collagenase type II (Worthington) in DMEM containing 3% (v/v) fetal bovine serum (FBS; Atlanta Biologicals) for 18 hours at 37° C. with gentle rocking. After digestion, the resultant cell solutions are to be filtered through 70 μm cell strainers.

Protocol for introducing shear to purify chondrocytes: (1) Take up cell solution into a sterile 10 mL syringe. (2) Attach the syringe to a microfluidic device with channels 75 μm-200 μm in diameter. (3) Slowly depress the syringe plunger so that the cell solution flows through the microfluidic device and into a conical tube reservoir. (4) Once the syringe has been fully depressed, inject another 20 mL of DMEM into the microfluidic device. (5) Wash the processed cell solution twice with wash medium and count the remaining cells.

### EXAMPLE 9

### Impact/Compression

Example 9 describes methods of using an impact/compression to select cells based on stiffness properties. Example 9 shows a protocol by which to purify articular chondrocytes with the application of compression/impact. Cell isolation: Juvenile bovine articular chondrocytes are to be harvested from the patellofemoral surfaces of bovine stifle joints. Articular cartilage is to be minced into approximately 1 mm³ pieces and washed and centrifuged (500 G for 5 minutes) three times with Dulbecco's Modified Eagle Medium containing 4.5 g/L glucose and GlutaMAX (DMEM; Gibco) and 2% (v/v) penicillin/streptomycin/fungizone (PSF; BD Biosciences). Minced tissue is to be digested in 0.2% (w/v) collagenase type II (Worthington) in DMEM containing 3% (v/v) fetal bovine serum (FBS; Atlanta Biologicals) for 18 hours at 37° C. After digestion, the resultant cell solutions are to be filtered through 70 μm cell strainers, centrifuged (500 G for 5 minutes), and resuspended in blank DMEM.

Protocol for introducing impact/compression to purify chondrocytes: (1) Place approximately 50 mL cell solution in petri dishes. Add 20 glass beads of 0.25-0.5 mm to the petri dishes. (2) Submerge the paddle rotor into the petri dish and rotate it at 20-rpm for 3 minutes. (3) Remove the paddle rotor and pipette out the cell solution into conical tubes. (4) Wash the glass beads with 50 mL DMEM and place washing DMEM into conical tubes with the processed cell solutions. (5) Wash the processed cell solution twice with wash medium and count the remaining cells.

### EXAMPLE 10

Example 10 describes enhancing translatability of purified and expanded chondrocytes to engineer native-like neocartilage. The present invention is not limited to the methods or compositions described herein.

Chondrocytes were isolated from fetal sheep stifles, as fetal cells represent a highly-clinically relevant cell type for tissue engineering. First, ACK buffer treatment of primary (P0) chondrocytes decreased red blood cell contamination by 60% and increased neocartilage aggregate modulus (1.8-fold), shear modulus (1.3-fold), and tensile modulus (0.8-fold). Subsequently, seeding density optimization of expanded/redifferentiated (P3R) chondrocytes to 2 million cells/construct increased aggregate modulus (1.0-fold) and shear modulus (1.1-fold) further. Lastly, cytochalasin D treatment further increased neocartilage aggregate modulus to 400 kPa, on par with native cartilage, 9.6-fold over the untreated P0 control. ACK buffer- and cytochalasin D-treated P3R cells notably yielded neocartilage with compressive properties beyond that of P0 neocartilage and akin to native cartilage. These sequential studies allowed 4000-times fewer primary cells to be used to engineer robust neocartilage, specifically using 1000 primary cells per P3R construct versus 4,000,000 per P0 construct, greatly enhancing the clinical translatability of expanded chondrocytes for tissue engineering.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims.

In some embodiments, descriptions of the inventions described herein using the phrase “comprising” includes embodiments that could be described as “consisting of”, and as such the written description requirement for claiming one or more embodiments of the present invention using the phrase “consisting of” is met.

What is claimed is:

1. A method of enhancing a cell population, the method comprises:
a. obtaining a population of somatic cells;
b. subjecting the population of cells from (a) to a treatment that selects for cells that have pre-existing undesirable cytoskeletal characteristics;
c. isolating or removing the selected cells from (b) that have pre-existing undesirable cytoskeletal characteristics; and
d. isolating and retaining the remaining cell population from (b), enriched for cells without undesirable cytoskeletal characteristics,
wherein the methods can be repeated multiple times, alone or in combination with other treatments.

2. The method of claim 1, wherein the cells with pre-existing undesirable cytoskeletal characteristics comprise cells with weakened, fragmented, disrupted, or modified cytoskeletons, cells with cytoskeletons that are unable to remodel or have reduced remodeling ability, cells with cytoskeletal properties that are more susceptible to destruction by the method of claim 1, or a combination thereof.

3. The method of claim 1, wherein treating the cells to induce cell swelling or shrinking comprises one or more of the following:
a. adding a hypotonic solution, including ammonium chloride potassium (ACK) buffer;
b. adding a hypertonic solution;
c. performing freeze thaw cycles;
d. applying decompression of dissolved gases
e. applying a vacuum or negative pressure;
f. applying high frequency oscillations, including sonication, to induce cavitation; and/or
g. applying hydrostatic pressure.

4. The method of claim 1, wherein treating the cells to induce shearing or tension comprises one or more of the following:
a. fluid flow-induced shear;
b. fluid flow-induced tension;
c. opposing microfluidic flow;
d. forcing cells through a small filter/mesh or pathway/tunnel at high pressure; and/or
e. nebulizing the cell solution.

5. The method of claim 1, wherein treating the cells with impact or compression comprises one or more of the following:
a. forcing through a small filter/mesh or pathway/tunnel at high pressure;
b. applying mechanical compression; and/or
c. applying or inducing physical collisions.

6. The method of claim 1, wherein the method is for treating a subject comprises one or more of the following: direct use of cells; in vitro culture of cells comprising passaging in monolayer or in three-dimensional environment including suspension culture; tissue engineering using scaffold-free systems including self-assembly or using scaffold-based systems including natural and synthetic materials; cell transfer; tissue transfer; and/or grafting.

7. The method of claim 1, wherein the isolated, retained cells or tissues engineered/fabricated from the isolated, retained cells are subjected to treatment comprising one or more of the following: growth factors (including TGFI β superfamily); cytoskeleton modifying agents (including cytochalasin family); hormones (including triiodothyronine); toxic compounds (including staurosporine); molecules that act upstream in a signaling cascade (including Y27632); varying oxygen tensions (including hypoxia obtained via environmental or enzymatic means); crosslinking agents (including lysyl oxidase-like 2 protein); matrix degrading enzymes (including chondroitinase-ABC), matrix molecules (including link protein); and/or mechanical stimulation (including uniaxial tension, fluid flow-induced shear, or hydrostatic pressure).

8. A method of enhancing a cell population, the method comprises:
a. obtaining a population of somatic cells
b. subjecting the population of cells from (a) to a treatment to select for cells that have pre-existing undesirable membrane characteristics;
c. isolating or removing the selected cells form (b) that have pre-existing undesirable membrane characteristics; and
d. isolating and retaining the remaining cell population from (b), enriched for cells without undesirable membrane characteristics,
wherein the methods can be repeated multiple times, alone or in combination with other treatments.

9. The method of claim 8, wherein the cells with undesirable membrane characteristics comprise cells with reduced membrane surface area, cells with a disrupted or modified membrane, cells with membrane surface area unable to adjust to conformational changes, cells with membrane properties that render the cells more susceptible to destruction by the method of claim 8, or a combination thereof.

10. The method of claim 8 wherein treating the cells to induce cell swelling or shrinking comprises one or more of the following:
h. adding a hypotonic solution, including ammonium chloride potassium (ACK) buffer;
i. adding a hypertonic solution;
j. performing freeze thaw cycles;
k. applying decompression of dissolved gases
l. applying a vacuum or negative pressure;
m. applying high frequency oscillations including sonication, to induce cavitation; and/or
n. applying hydrostatic pressure.

11. The method of claim 8 wherein treating the cells to induce shearing or tension comprises one or more of the following:
a. fluid flow-induced shear;
b. fluid flow-induced tension;
c. opposing microfluidic flow;
d. forcing cells through a small filter/mesh or pathway/tunnel at high pressure; and/or
e. nebulizing the cell solution.

12. The method of claim 8, wherein treating the cells with impact or compression comprises one or more of the following:
a. forcing through a small filter/mesh or pathway/tunnel at high pressure;
b. applying mechanical compression; and/or
c. applying or inducing physical collisions.

13. The method of claim 8, wherein the method is for treating a subject comprises one or more of the following: direct use of cells; in vitro culture of cells comprising passaging in monolayer or in three-dimensional environment including suspension culture; tissue engineering using scaffold-free systems including self-assembly or using scaffold-based systems including natural and synthetic materials; cell transfer; tissue transfer; and/or grafting.

14. The method of claim 8, wherein the isolated, retained cells or tissues engineered/fabricated from the isolated, retained cells are subjected to treatment comprising one or more of the following: growth factors (including TGF β superfamily); cytoskeleton modifying agents (including cytochalasin family); hormones (including triiodothyronine); toxic compounds (including staurosporine); molecules that act upstream in a signaling cascade (including Y27632); varying oxygen tensions (including hypoxia obtained via environmental or enzymatic means); crosslinking agents (including lysyl oxidase-like 2 protein); matrix degrading enzymes (including chondroitinase-ABC), matrix molecules (including link protein); and/or mechanical stimulation (including uniaxial tension, fluid flow-induced shear, or hydrostatic pressure).

15. A method of enhancing a cell population, the method comprises:
a. obtaining a population of somatic cells,
b. subjecting the population of cells from (a) to a treatment to select for cells that have pre-existing altered stiffness;
c. isolating or removing the selected cells from (b) that have pre-existing altered stiffness; and
d. isolating and retaining the remaining cell population from (b), enriched for cells without altered stiffness,
wherein the methods can be repeated multiple times, alone or in combination with other treatments.

16. The method of claim 15, wherein the cells with pre-existing altered stiffness characteristics comprise cells with reduced overall stiffness, with increased overall stiffness, cells with stiffness which varies depending on the region of the cell tested, cells with reduced pliability, cells with stiffness properties that render the cells more susceptible to destruction by the method of claim 15, or a combination thereof.

17. The method of claim 15, wherein treating the cells to induce cell swelling or shrinking comprises one or more of the following:
a. adding a hypotonic solution including ammonium chloride potassium (ACK) buffer;
b. adding a hypertonic solution;
c. performing freeze thaw cycles;
d. applying decompression of dissolved gases
e. applying a vacuum or negative pressure;
f. applying high frequency oscillations including sonication, to induce cavitation; and/or
g. applying hydrostatic pressure.

18. The method of claim 15, wherein treating the cells to induce shearing or tension comprises one or more of the following:
a. fluid flow-induced shear;
b. fluid flow-induced tension;
c. opposing microfluidic flow;
d. forcing cells through a small filter/mesh or pathway/tunnel at high pressure; and/or
e. nebulizing the cell solution.

19. The method of claim 15, wherein treating the cells with impact or compression comprises one or more of the following:
a. forcing through a small filter/mesh or pathway/tunnel at high pressure;
b. applying mechanical compression; and/or
c. applying or inducing physical collisions.

20. The method of claim 15, wherein the method is for treating a subject comprises one or more of the following: direct use of cells; in vitro culture of cells comprising passaging in monolayer or in three-dimensional environment including suspension culture; tissue engineering using scaffold-free systems including self-assembly or using scaffold-based systems including natural and synthetic materials; cell transfer; tissue transfer; and/or grafting.

21. The method of claim 15, wherein the isolated, retained cells or tissues engineered/fabricated from the isolated, retained cells are subjected to treatment comprising one or more of the following: growth factors (including TGF β superfamily); cytoskeleton modifying agents (including cytochalasin family); hormones (including triiodothyronine); toxic compounds (including staurosporine); molecules that act upstream in a signaling cascade (including Y27632); varying oxygen tensions (including hypoxia obtained via environmental or enzymatic means); crosslinking agents (including lysyl oxidase-like 2 protein); matrix degrading enzymes (including chondroitinase-ABC), matrix molecules (including link protein); and/or mechanical stimulation (including uniaxial tension, fluid flow-induced shear, or hydrostatic pressure).
